(19) United States
(12) Patent Application Publication
(10) Pub. No.: US 2026/0314945 A1
(43) Pub. Date: Oct. 15, 2026

(54) WEARABLE DEVICE

(71) Applicant: TERUMO KABUSHIKI KAISHA, Tokyo (JP)

(72) Inventor: Masaomi IMAI, Chuo-shi (JP)

(73) Assignee: TERUMO KABUSHIKI KAISHA, Tokyo (JP)

(21) Appl. No.: 19/465,534

(22) Filed: Jan. 30, 2026

Related U.S. Application Data
(63) Continuation of application No. PCT/JP2024/026847, filed on Jul. 26, 2024.

(30) Foreign Application Priority Data
Aug. 1, 2023 (JP) 2023-125749

(51) Int. Cl. A61B 5/024; A61B 5/00; A61B 5/0507; A61B 5/053; A61B 7/04

(52) U.S. Cl. CPC A61B 5/02427; A61B 5/0507; A61B 5/053; A61B 5/681; A61B 7/04; A61B 2562/046; A61B 2562/227

(57) ABSTRACT

A wearable device according to the present disclosure includes: a sensor main body including an electrode that detects a living-body’s electrical characteristic and a detection portion that detects other living-body information than the living-body’s electrical characteristic; and an attachment portion that is attached to and detached from the sensor main body and is attached to a living-body’s surface, in which the attachment portion includes a conduction portion making the sensor-main-body’s electrode and the living-body’s surface conductive with each other when the sensor-main-body’s electrode and the living-body’s surface come into contact with each other, and the attachment portion does not act as a blockage between the sensor-main-body’s detection portion and the living-body’s surface in a conduction state where the attachment portion fitted with the sensor main body is attached to the living-body’s surface and the sensor-main-body’s electrode and the living-body’s surface are conductive with each other via the attachment-portion’s conduction portion.

### CROSS-REFERENCES TO RELATED APPLICATIONS

This application is a continuation of International Patent Application No. PCT/JP2024/026847 filed on Jul. 26, 2024, which claims priority to Japanese Patent Application No. 2023-125749 filed on Aug. 1, 2026, the entire content of both of which is incorporated herein by reference.

### TECHNOLOGICAL FIELD

The present disclosure generally relates to a wearable device.

### Background Discussion

Conventionally, a wearable device is known that is configured to be attached to a surface of a living body and includes an electrode configured to detect an electrical characteristic of the living body on a surface that comes into contact with the surface of the living body. JP 2016-214641 A discloses a living body information measurement device serving as a wearable device of this type. The living body information measurement device described in JP 2016-214641 A includes a case portion that houses a device main body including a living body information detection portion that detects living body information of a user, a band portion used to fit the case portion to the user, and an electrode disposed on a surface of the case portion

or the band portion, the surface being allowed to come into contact with a body of the user. In addition, the living body information detection portion described in JP 2016-214641 A includes an electrocardiogram measurement portion and a pulse wave detection portion.

### SUMMARY

In the living body information measurement device serving as the wearable device described in JP 2016-214641 A, the electrode that comes into contact with a surface of a living body is likely to deteriorate due to contact with the surface of the living body or adhesion associated with sweat that is present on the surface of the living body, for example. In the living body information measurement device described in JP 2016-214641 A, it may be required to wholly replace the living body information measurement device including the living body information detection portion due to deterioration of the electrode as described above. That is, in the living body information measurement device described in JP 2016-214641 A, deterioration of the electrode may determine the life cycle of the entire device, even when it is not necessary to replace components other than the electrode (for example, a control portion or the pulse wave detection portion) in the device main body serving as the sensor main body. Therefore, in the living body information measurement device described in JP 2016-214641 A, there is still room for improvement from a viewpoint of reusability of the sensor main body.

In addition, the living body information detection portion described in JP 2016-214641 A includes the pulse wave detection portion serving as a detection portion configured to detect other living body information than the electrical characteristic of the living body in addition to the electrocardiogram measurement portion that utilizes the electrical characteristic of the living body, which is to be provided from the electrode. In such a case, it is preferable to improve the reusability of the sensor main body described above while suppressing a decrease in detection accuracy by the detection portion.

The wearable device configured to achieve both improvement in reusability of a sensor main body and suppression of a decrease in detection accuracy by a detection portion configured to detect other living body information than an electrical characteristic of a living body.

A wearable device serving as a first aspect of the present disclosure is (1) a wearable device including:
a sensor main body including an electrode configured to detect an electrical characteristic of a living body and a detection portion configured to detect other living body information than the electrical characteristic of the living body; and
an attachment portion configured to be attached to and detached from the sensor main body and configured to be attached to a surface of the living body, in which
the attachment portion includes a conduction portion that makes the electrode of the sensor main body and the surface of the living body conductive with each other when the electrode of the sensor main body and the surface of the living body come into contact with each other, and
the attachment portion is configured to not act as a blockage between the detection portion of the sensor main body and the surface of the living body in a conduction state where the attachment portion fitted with the sensor main body is attached to the surface of the living body and the electrode of the sensor main body and the surface of the living body are conductive with each other via the conduction portion of the attachment portion.

A wearable device serving as one embodiment of the present disclosure is (2),
the wearable device described above in (1), in which
   the attachment portion includes a back surface that covers the surface of the living body in the conduction state and a front surface lying opposite to the back surface,
   the sensor main body is configured to be detached from and attached to the attachment portion on a side on which the front surface of the attachment portion is present,
   an opening portion penetrating from the side on which the front surface is present to a side on which the back surface is present is formed on the attachment portion, and
   the detection portion of the sensor main body is configured to detect the living body information through the opening portion of the attachment portion in the conduction state.

A wearable device serving as another one embodiment of the present disclosure is
(3) the wearable device described above in (2), in which the sensor main body is inserted into the opening portion from the side on which the front surface of the attachment portion is present in the conduction state.

A wearable device serving as still another one embodiment of the present disclosure is
(4) the wearable device described above in (3), in which the sensor main body protrudes from the back surface of the attachment portion through the opening portion of the attachment portion in the conduction state.

A wearable device serving as still another one embodiment of the present disclosure is (5) the wearable device described above in any one of (2) to (4), in which the conduction portion of the attachment portion includes:
a front surface exposure portion that is exposed on the front surface and is configured to come into contact with the electrode of the sensor main body; and
a back surface exposure portion that is exposed on the back surface and is configured to come into contact with the surface of the living body.

A wearable device serving as still another one embodiment of the present disclosure is
(6) the wearable device described above in (5), in which
a housing recess portion configured to house the sensor main body is formed on the front surface of the attachment portion, and
the front surface exposure portion of the conduction portion is exposed on a bottom surface of the housing recess portion on the front surface.

A wearable device serving as still another one embodiment of the present disclosure is
(7) the wearable device described above in (5) or (6), in which
a housing recess portion configured to house the sensor main body is formed on the front surface of the attachment portion, and
the front surface exposure portion of the conduction portion is exposed on a side surface of the housing recess portion on the front surface.

A wearable device serving as still another one embodiment of the present disclosure is
(8) the wearable device described above in any one of (5) to (7), in which
the attachment portion is a band body extending in circumferential directions of an arm and configured to be attached to the arm, and
the back surface exposure portion and the front surface exposure portion of the conduction portion are disposed at positions overlapping each other in extending directions of the band body.

A wearable device serving as still another one embodiment of the present disclosure is
(9) the wearable device described above in any one of (5) to (7), in which
the attachment portion is a band body extending in circumferential directions of an arm and configured to be attached to the arm, and
the back surface exposure portion and the front surface exposure portion of the conduction portion are disposed at positions not overlapping each other in extending directions of the band body.

A wearable device serving as still another one embodiment of the present disclosure is
(10) the wearable device described above in (9), in which the conduction portion includes a wiring portion that electrically couples the back surface exposure portion and the front surface exposure portion to each other and is embedded in the band body without being exposed to outside.

A wearable device serving as still another one embodiment of the present disclosure is
(11) the wearable device described above in any one of (1) to (10), in which
the sensor main body includes a plurality of the electrodes, and
the attachment portion includes a plurality of the conduction portions respectively corresponding to the plurality of electrodes.

A wearable device serving as still another one embodiment of the present disclosure is
(12) the wearable device described above in any one of (1) to (11), in which the detection portion of the sensor main body is a sensor configured to detect an electromagnetic wave or a sound wave from the living body.

A wearable device serving as still another one embodiment of the present disclosure is
(13) the wearable device described above in (12), in which the detection portion of the sensor main body is an optical pulse wave sensor.

According to the present disclosure, it is possible to provide a wearable device configured to achieve both improvement in reusability of a sensor main body and suppression of a decrease in detection accuracy by a detection portion configured to detect other living body information than an electrical characteristic of a living body.

According to another aspect, a wearable device comprises a base body that includes a housing fitting portion and an elongated band configured to encircle a portion of a living body so that a surface of the base body faces towards and is in contact with a surface of the living body. The housing fitting portion includes a recess surrounded by a bottom wall portion of the housing fitting portion and a side wall portion of the housing fitting portion, with the side wall portion of the housing fitting portion projecting away from the bottom wall portion of the housing fitting portion towards an open upper end of the recess. A sensor main body is configured to be positioned in the recess of the housing fitting portion, and the sensor main body includes a top wall, a bottom wall and a side wall extending between the top wall and the bottom wall of the sensor main body so that when the sensor main body is positioned in the recess of the housing fitting portion, the bottom wall of the sensor main body faces the bottom wall portion of the housing fitting portion and the side wall of the sensor main body faces the side wall portion of the housing fitting portion;. The sensor main body includes an electrode configured to detect an electrical characteristic of a living body and a detection portion different from the electrode and configured to detect living body information other than the electrical characteristic of the living body. The electrode has an exposed portion that is exposed on the bottom wall of the sensor main body or on the side wall of the sensor main body, and the side wall portion of the housing fitting portion or the bottom wall portion of the housing fitting portion includes a conduction portion configured to contact the exposed portion of the electrode of the sensor main body when the sensor main body is positioned in the recess to provide conductivity between the electrode of the sensor main body and the surface of the living body when the wearable device is worn on the living body. The base body is configured to not act as a blockage between the detection portion of the sensor main body and the surface of the living body in a conduction state in which the sensor main body is positioned in the recess and the surface of the base body is in contact with the surface of the living body.

### BRIEF DESCRIPTION OF THE DRAWINGS

FIG. 1 is a perspective view when a wearable device serving as one embodiment is viewed from a side on which its front surface is present, illustrating a state where a sensor main body is fitted to a base body;

FIG. 2 is a perspective view when the wearable device illustrated in FIG. 1 is viewed from a side on which its back surface is present;

FIG. 3 is a front view of the wearable device illustrated in FIG. 1;

FIG. 4 is a view illustrating a use state of the wearable device illustrated in FIG. 1;

FIG. 5 is a view illustrating the use state of the wearable device illustrated in FIG. 1;

FIG. 6 is an exploded perspective view when a state where the base body and the sensor main body of the wearable device illustrated in FIG. 1 are separated from each other is viewed from the side on which the front surface is present;

FIG. 7 is an exploded perspective view when the wearable device illustrated in FIG. 6 is viewed from the side on which the back surface is present;

FIG. 8 is a cross-sectional view of the wearable device, which is taken at a position along the section line 8-8 illustrated in FIG. 3;

FIG. 9 is a cross-sectional view of the wearable device, which is taken at a position along the section line 9-9 illustrated in FIG. 3;

FIG. 10 is a perspective view when a wearable device serving as another one embodiment is viewed from the side on which its front surface is present, illustrating a state where the sensor main body is fitted to the base body;

FIG. 11 is an exploded perspective view when a state where the base body and the sensor main body of the wearable device illustrated in FIG. 10 are separated from each other is viewed from the side on which the front surface is present;

FIG. 12 is an exploded perspective view when the wearable device illustrated in FIG. 11 is viewed from the side on which the back surface is present;

FIG. 13 is a side view of a base main body of the base body, a base cover of the base body, and the sensor main body illustrated in FIG. 11;

FIG. 14 is a view illustrating the use state of the wearable device illustrated in FIG. 10;

FIG. 15 is an exploded perspective view of a wearable device serving as still another one embodiment;

FIG. 16 is a cross-sectional view of a fitting portion of the wearable device illustrated in FIG. 15; and

FIG. 17 is a view illustrating a contact state of conduction portions of the base body and electrodes of the sensor main body in the wearable device illustrated in FIG. 15.

### DETAILED DESCRIPTION

Hereinafter, embodiments of a wearable device according to the present disclosure will be described by way of example with reference to the drawings. In the drawings, the same components are denoted by the same reference signs.

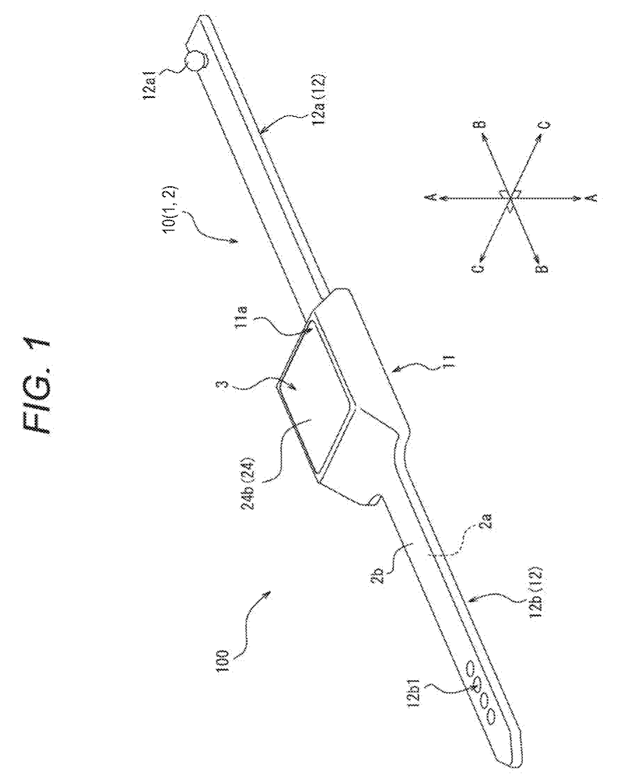

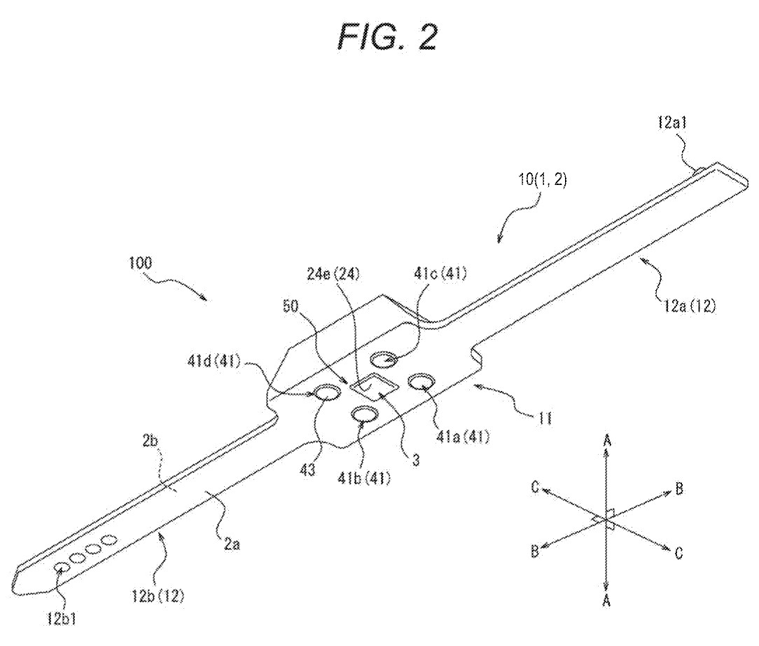

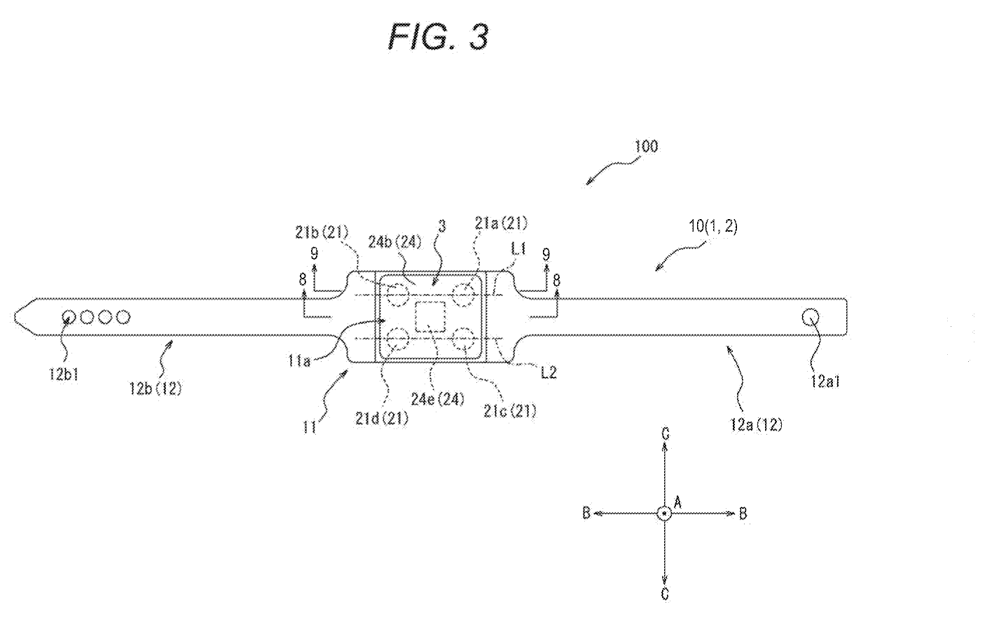

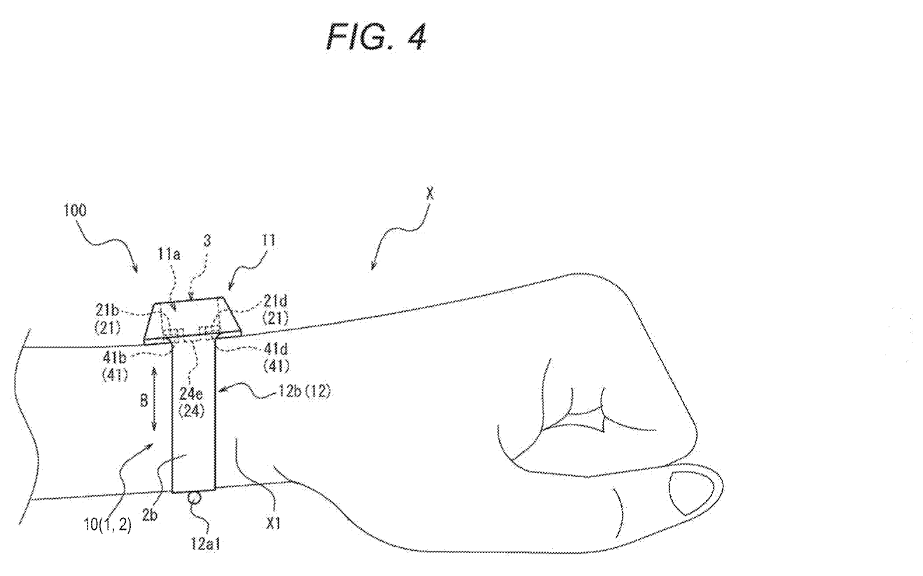

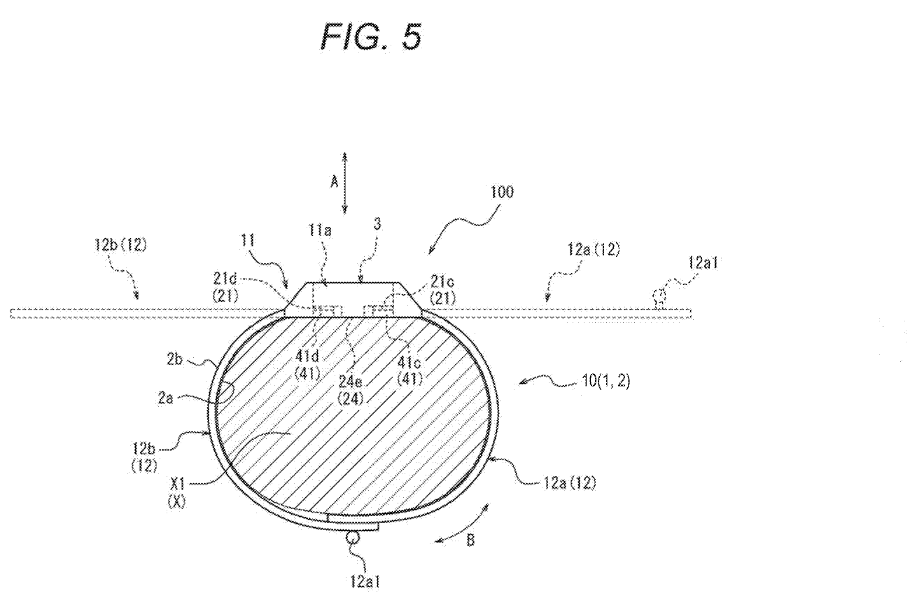

FIGS. 1 and 2 are perspective views of a wearable device 100 serving as one embodiment of the wearable device according to the present disclosure. As illustrated in FIG. 1, the wearable device 100 includes an attachment portion 1 configured to be attached to a surface of a living body and a sensor main body 3 configured to be detached from and attached to the attachment portion 1. The attachment portion 1 according to the present embodiment is a base body 2 that is to be attached to the surface of the living body and is to be interposed between the surface of the living body and the sensor main body 3. FIG. 1 is the perspective view when the wearable device 100 in a state where the sensor main body 3 is fitted to the base body 2 is viewed from a side on which its front surface is present. FIG. 2 is the perspective view when the wearable device 100 in the state where the sensor main body 3 is fitted to the base body 2 is viewed from a side on which its back surface is present. In addition, FIG. 3 is a front view of the wearable device 100 in the state where the sensor main body 3 is fitted to the base body 2. FIGS. 4 and 5 are views illustrating a use state of the wearable device 100.

A method of using the wearable device 100 will first be described with reference to FIGS. 4 and 5.

The wearable device 100 is attached onto the surface of the living body for use. As illustrated in FIGS. 4 and 5, the wearable device 100 according to the present embodiment is configured to be used when wound around an arm X of a user. More specifically, the wearable device 100 according to the present embodiment includes a band body (band) 10 serving as the base body 2. As illustrated in FIGS. 4 and 5, the band body 10 is allowed to extend in circumferential directions of the arm X of the user, and is attached to the arm X in a state of being wound around or encircling the arm X.

Although FIGS. 4 and 5 illustrate an example where the wearable device 100 is wound around a wrist X1 of the arm X of the user, the wearable device 100 is not limited in attachment position to the wrist X1. The wearable device 100 may be wound around a forearm of the arm X of the user for use, for example. In addition, the wearable device 100 may be wound around the surface of the living body at a position other than the arm X, such as a leg of the user for use, for example.

Furthermore, although the wearable device 100 according to the present embodiment includes the band body 10 serving as the base body 2, which is to be wound around the surface of the living body for use, the base body 2 is not limited to the band body 10. The base body 2 may include, for example, an adhesion surface that is configured to be adhered to the surface of the living body, and may be adhered to the surface of the living body and attached to the surface of the living body. In such a case, the surface of the living body to which the base body 2 is attached may be, for example, a position different from the arm X or the leg, such as the chest.

In the present embodiment, an example of the method of using the wearable device 100, in which the wearable device 100 is wound around the arm X of the user, will now be described herein.

As illustrated in FIGS. 4 and 5, the wearable device 100 is configured to acquire living body information in the state of being wound around the wrist X1 of the arm X of the user. More specifically, the wearable device 100 is configured to acquire living body information that is based on an electrical characteristic of the living body and other living body information that is not based on the electrical characteristic of the living body.

Examples of the living body information that is based on the electrical characteristic of the living body include, for example, living body impedance detected when electricity is allowed to flow into the living body and electrocardiographic information related to movements of a heart, which is acquired when an electrical signal flowing in the living body is detected. In addition, examples of other living body information that is not based on the electrical characteristic of the living body include, for example, pulse wave information and blood pressure information. These are living body information acquired without allowing electricity to flow through the living body or detecting an electric signal flowing through the living body. The wearable device 100 according to the present embodiment is configured to acquire living body impedance serving as living body information that is based on the electrical characteristic of the living body. In addition, the wearable device 100 according to the present embodiment is configured to acquire pulse wave information serving as other living body information that is not based on the electrical characteristic of the living body.

As described above, the wearable device 100 according to the present embodiment is configured to acquire living body impedance and pulse wave information of the user when wound around the arm X of the user for use.

The wearable device 100 according to the present embodiment is configured to acquire living body impedance from the wrist X1 of the user to make it possible to measure an amount of moisture in the wrist X1 of the user. It is known that, when a cardiac function of a living body is deteriorated, edema in which moisture accumulates in or under skin occurs. With the wearable device 100, monitoring an amount of moisture in the wrist X1 of the user who is a patient of heart disease, for example, makes it possible to detect deterioration in cardiac function of the user at an early stage.

In addition, the wearable device 100 according to the present embodiment is configured to acquire pulse wave information from the wrist X1 of the user to make it possible to perform monitoring in heartbeat of the user in addition to the amount of moisture in the user. Therefore, it is possible to consider deterioration in cardiac function of the user from a viewpoint of variation in heartbeat in addition to a viewpoint of variation in amount of moisture.

The wearable device 100 may be used, for example, in a state of being constantly attached to the user for a certain period of time such as several hours or several days. Doing so makes it possible to constantly monitor a state of the cardiac function of the user. Thereby, it is possible to detect deterioration in cardiac function of the user at an early stage. However, the wearable device 100 may also be temporarily attached to the user and may be used for a purpose of grasping or understanding a state of the cardiac function of the user at a predetermined timing.

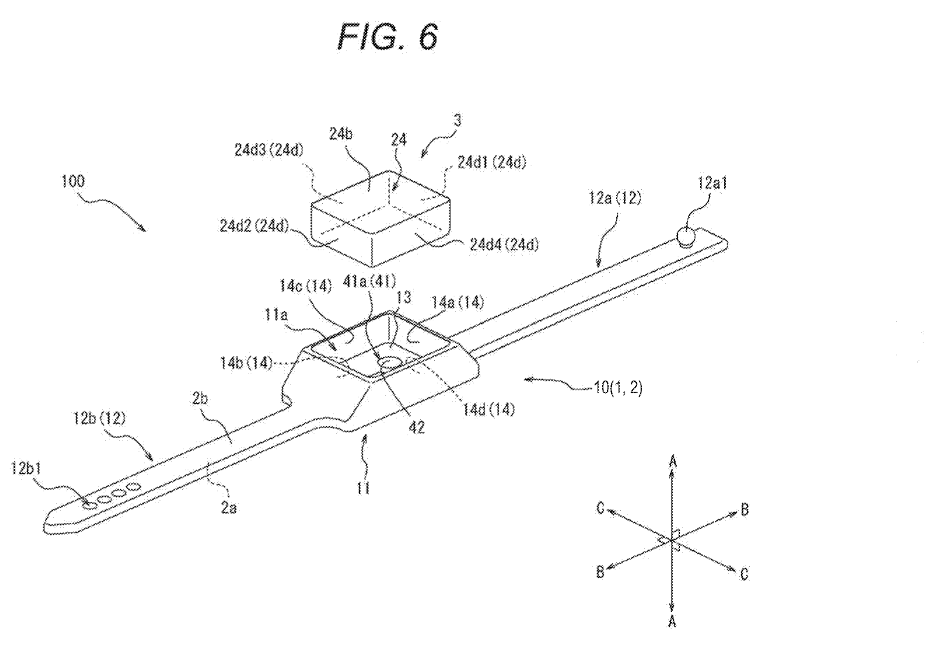

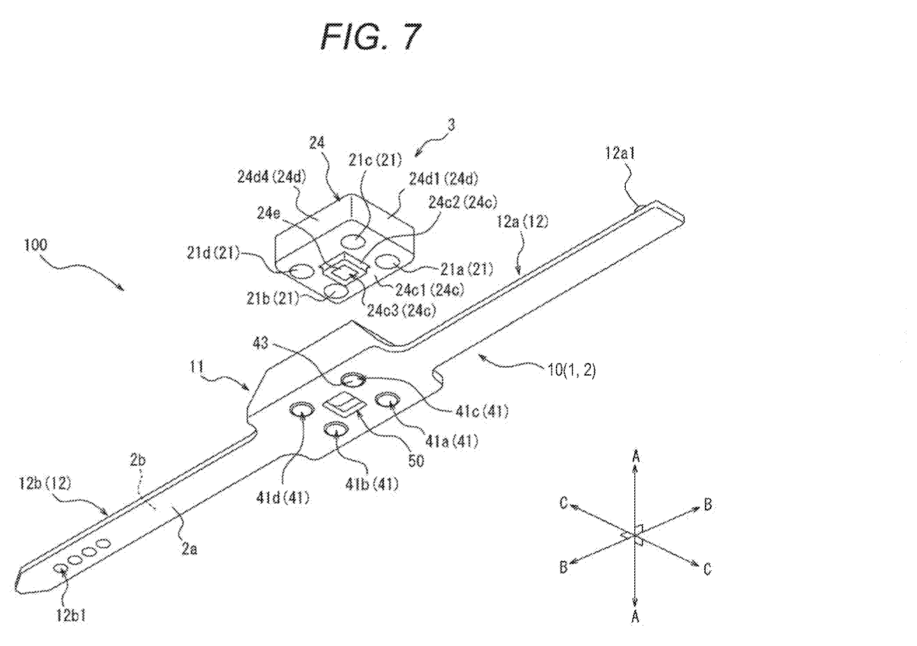

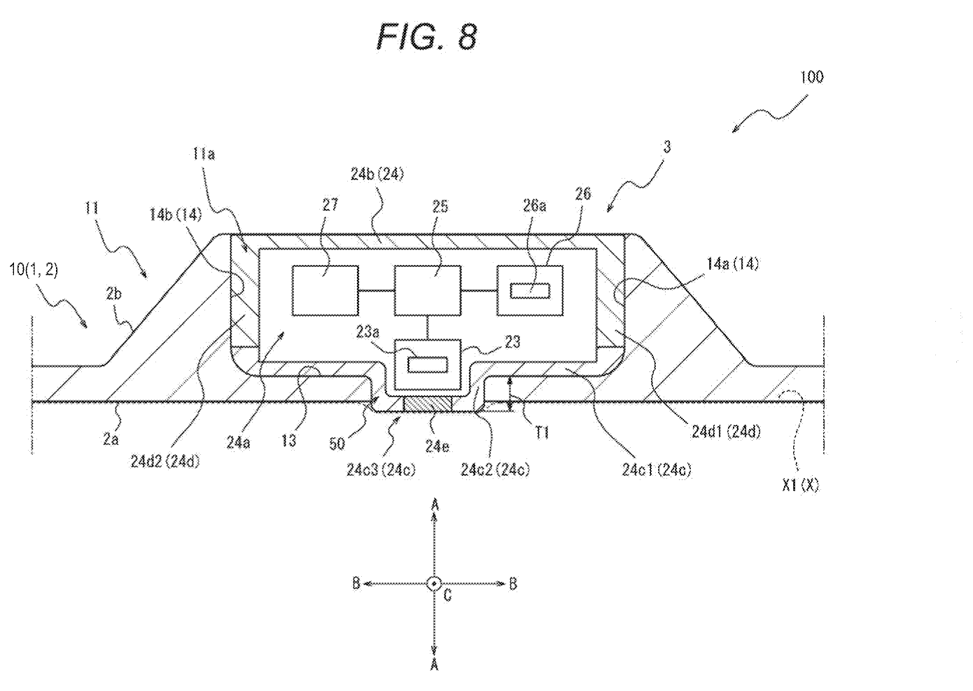

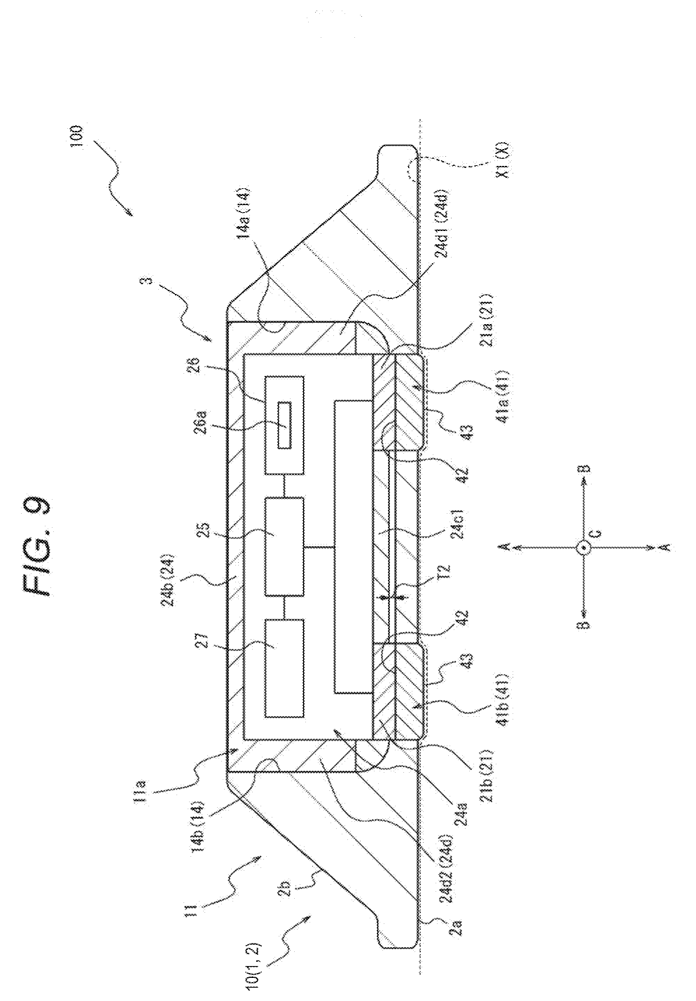

The wearable device 100 will now be described herein in detail with reference to FIGS. 1 - 9. FIGS. 6 and 7 are exploded perspective views of the wearable device 100, illustrating that the base body 2 and the sensor main body 3 of the wearable device 100 are separated from each other or separable from each other. FIG. 6 is the exploded perspective view when the wearable device 100 in a state where the base body 2 and the sensor main body 3 are separated from each other is viewed from the side on which the front surface is present. FIG. 7 is the exploded perspective view when the wearable device 100 in the state where the base body 2 and the sensor main body 3 are separated from each other is viewed from the side on which the back surface is present. FIG. 8 is a cross-sectional view of the wearable device 100, which is taken at a position along the section line 8-8 illustrated in FIG. 3. FIG. 9 is a cross-sectional view of the wearable device 100, which is taken at a position along the section line 9-9 illustrated in FIG. 3. In FIGS. 8 and 9, a broken line indicates, for convenience of description, a position of an outer surface of the wrist X1 of the arm X of the user, to which the wearable device 100 is attached.

As described above, the wearable device 100 includes the base body 2 and the sensor main body 3. The base body 2 is configured to be attached to the surface of the living body. The sensor main body 3 is configured to be detached from and attached to the base body 2.

The sensor main body 3 includes electrodes 21 configured to detect the electrical characteristic of the living body and a detection portion 23 (see FIG. 8) configured to detect other living body information than the electrical characteristic of the living body. The sensor main body 3 according to the present embodiment is configured to acquire living body impedance based on the electrical characteristic of the living body, which the electrodes 21 detect. The detection portion 23 may be a sensor configured to detect an electromagnetic wave or a sound wave from the living body. The detection portion 23 according to the present embodiment is an optical pulse wave sensor utilizing photoplethysmography. The sensor main body 3 according to the present embodiment is configured to acquire pulse wave information (pulse wave signal) based on the optical pulse wave sensor serving as the detection portion 23.

The base body 2 includes conduction portions 41. The conduction portions 41 are configured to make the electrodes 21 of the sensor main body 3 and the surface of the living body conductive with each other when the electrodes 21 of the sensor main body 3 and the surface of the living body come into contact with each other, respectively. As illustrated in FIG. 9, the sensor main body 3 is configured to be fitted to the base body 2 to allow the electrodes 21 to come into contact with the conduction portions 41, respectively. As described above, the base body 2 is attached to the surface of the living body in a state where the conduction portions 41 of the base body 2 and the electrodes 21 of the sensor main body 3 are in contact with each other and are conductive with each other. As described above, the base body 2 according to the present embodiment is attached to the wrist X1 of the user in the state of being wound around the wrist X1 (see FIGS. 4 and 5). At this time, the conduction portions 41 of the base body 2 come into contact with the outer surface of the wrist X1, which serves as the surface of the living body. That is, the base body 2 is attached to the surface of the living body, and a conduction state is attained where the electrodes 21 of the sensor main body 3 and the surface of the living body are conductive with each other via the conduction portions 41 of the base body 2. This state will be hereinafter simply referred to as the "conduction state" for convenience of description.

The base body 2 is configured to not act as a blockage between the detection portion 23 of the sensor main body 3 and the surface of the living body in the conduction state. Although details will be described later, the base body 2 according to the present embodiment is formed with an opening portion 50 (see FIG. 7, for example). As illustrated in FIG. 9, the detection portion 23 of the sensor main body 3 is configured to detect pulse wave information serving as other living body information than the electrical characteristic of the living body through the opening portion 50 of the base body 2.

As described above, in the wearable device 100, the sensor main body 3 is configured to be detached from and attached to the base body 2. In addition, the electrodes 21 of the sensor main body 3 are configured to not be in direct contact with the surface of the living body but configured to be conductive with the surface of the living body via the conduction portions 41 of the base body 2. That is, the sensor main body 3 has no electrode that comes into direct contact with and that allows electricity to flow into tissue of the living body for conduction. Therefore, it is possible to suppress deterioration of the electrodes 21 of the sensor main body 3 due to contact with the surface of the living body or adhesion of sweat that is present on the surface of the living body, for example. On the other hand, although the conduction portions 41 of the base body 2 may be deteriorated due to contact with the surface of the living body or adhesion of sweat that is present on the surface of the living body, for example, the sensor main body 3 may be removed from the base body 2, and only the base body 2 may be solely replaced in such a situation. That is, it is possible to reuse the sensor main body 3 including various electronic components. Doing so makes it possible to improve reusability of the sensor main body 3 in the wearable device 100.

In addition, the base body 2 is configured to not act as a blockage between the detection portion 23 of the sensor main body 3 and the surface of the living body in the conduction state. Therefore, even in a configuration where the base body 2 is interposed between the sensor main body 3 and the surface of the living body, it is possible to suppress a decrease in detection accuracy of living body information by the detection portion 23. The detection portion 23 does not allow electricity to flow into the surface of the living body. Therefore, even if a portion related to detection of living body information by the detection portion 23 is not blocked by the base body 2 and directly comes into contact with skin serving as the surface of the living body or adhered with sweat, the portion is less likely to deteriorate. Although details will be described later, in the present embodiment, a detection window portion 24e of a housing 24 of the sensor main body 3 is configured to come into contact with the outer surface of the wrist X1, which serves as the surface of the living body, through the opening portion 50 (see FIG. 9).

As described above, the wearable device 100 is configured to achieve both improvement in reusability of the sensor main body 3 and suppression of a decrease in detection accuracy by the detection portion 23 configured to detect other living body information than the electrical characteristic of the living body.

The wearable device 100 according to the present embodiment will now be described herein in more detail. In a state where the wearable device 100 in a state where the sensor main body 3 is fitted to the base body 2 (see FIGS. 1 - 3, for example) is attached to the surface of the living body (see FIGS. 4 and 5), in the present embodiment, the surface serving as a side on which the surface of the living body is present will be hereinafter referred to as a “back surface”, and the surface lying opposite to the back surface will be hereinafter referred to as a “front surface”, for convenience of description. In addition, in the wearable device 100, a direction heading from the side on which the back surface is present to the side on which the front surface is present and a direction heading from the side on which the front surface is present to the side on which the back surface is present will be referred to as “thickness directions A”, for convenience of description. Furthermore, in the present embodiment, in the wearable device 100, directions orthogonal to the thickness directions A, in which the band body 10 serving as the base body 2 extends, will be referred to as “longitudinal directions B”. In addition, in the present embodiment, in the wearable device 100, directions orthogonal to the thickness directions A and the longitudinal directions B will be referred to as “width directions C”.

As illustrated in FIGS. 8 and 9, for example, the sensor main body 3 according to the present embodiment includes the housing 24, a control portion 25, a communication portion 26, and a rechargeable battery 27 in addition to the electrodes 21 and the detection portion 23 described above.

As illustrated in FIG. 7, the sensor main body 3 according to the present embodiment includes a plurality of the electrodes 21. Specifically, the sensor main body 3 according to the present embodiment includes four electrodes 21. The four electrodes 21 according to the present embodiment include a pair of electrodes 21a and 21b and another pair of electrodes 21c and 21d.

The pair of electrodes 21a and 21b are utilized to acquire living body impedance of the user. The other pair of electrodes 21c and 21d may also be utilized, for example, to acquire living body impedance of the user, similar to the pair of electrodes 21a and 21b. In addition, the other pair of electrodes 21c and 21d may further be used, for example, as application electrodes that allow an electric current to flow into the living body. Furthermore, the other pair of electrodes 21c and 21d may further be used, for example, to correct a measurement value of living body impedance to be acquired through the pair of electrodes 21a and 21b. As described above, the other pair of electrodes 21c and 21d may further be utilized to improve measurement accuracy of a measurement value of living body impedance to be acquired through the pair of electrodes 21a and 21b. In addition, the sensor main body 3 may have a configuration that does not include the other pair of electrodes 21c and 21d. The pair of electrodes 21a and 21b according to the present embodiment will be hereinafter referred to as a "pair of detection electrodes 21a and 21b", and the other pair of electrodes 21c and 21d according to the present embodiment will be hereinafter referred to as a "pair of auxiliary electrodes 21c and 21d", for convenience of description. In addition, the four electrodes 21a to 21d are simply referred to as the “electrodes 21” unless otherwise distinguished.

The electrodes 21 are, for example, dry electrodes including, for example, rubber or metal. However, it is preferable that the electrodes 21 include metal having high conductivity.

The electrodes 21 according to the present embodiment are exposed outside of the housing 24 described later.

In addition, as illustrated in FIG. 3, the pair of detection electrodes 21a and 21b according to the present embodiment are disposed side by side in the longitudinal directions B. The expression that the pair of detection electrodes 21a and 21b are “disposed side by side in the longitudinal directions B”, means that, in a plan view when the wearable device 100 is viewed in one of the thickness directions A (see FIG. 3), at least one virtual straight line (for example, a virtual straight line L1 illustrated in FIG. 3) that passes through the pair of detection electrodes 21a and 21b and is parallel to the longitudinal directions B is present. Doing so makes it possible to allow the pair of detection electrodes 21a and 21b to come into contact with the user at substantially equal positions in length directions of the arm X of the user and at different positions in the circumferential directions of the arm X of the user. Thereby, the pair of detection electrodes 21a and 21b are able to detect living body impedance in one cross section of the arm X of the user, which is substantially orthogonal to the length directions of the arm X. Therefore, it becomes easy to monitor a change in amount of moisture in the arm X of the user in association with a change in outer diameter of the arm due to edema, for example.

Furthermore, as illustrated in FIG. 3, the pair of auxiliary electrodes 21c and 21d according to the present embodiment are also disposed side by side in the longitudinal directions B. The expression that the pair of auxiliary electrodes 21c and 21d are “disposed side by side in the longitudinal directions B” means that, in a plan view when the wearable device 100 is viewed in one of the thickness directions A (see FIG. 3), at least one virtual straight line (for example, a virtual straight line L2 illustrated in FIG. 3) that passes through the pair of auxiliary electrodes 21c and 21d and is parallel to the longitudinal directions B is present.

Although, in the present embodiment, the pair of detection electrodes 21a and 21b and the pair of auxiliary electrodes 21c and 21d according to the present embodiment are disposed at different positions in the width directions C, the present disclosure is not limited to this configuration. The pair of detection electrodes 21a and 21b and the pair of auxiliary electrodes 21c and 21d may be disposed side by side in the longitudinal directions B. That is, the four electrodes 21a to 21d may be disposed side by side in the longitudinal directions B.

Furthermore, although, in the present embodiment, the pair of detection electrodes 21a and 21b and the pair of auxiliary electrodes 21c and 21d are disposed side by side in the longitudinal directions B, the present disclosure is not limited to this configuration. The pair of detection electrodes 21a and 21b and the pair of auxiliary electrodes 21c and 21d may be respectively disposed side by side in the width directions C at identical positions or different positions in the longitudinal directions B. However, as described above, it is preferable that the pair of detection electrodes 21a and 21b are disposed side by side in the longitudinal directions B.

The detection portion 23 according to the present embodiment is an optical pulse wave sensor utilizing photoplethysmography. The detection portion 23 according to the present embodiment includes an optical transmission/reception portion 23a configured to transmit and receive an optical signal through the detection window portion 24e of the housing 24 described later.

The housing 24 is an exterior member of the sensor main body 3. The housing 24 according to the present embodiment is a box body that includes resin, that has a rectangular shape, and that defines an internal space 24a. In the present embodiment, the detection portion 23, the control portion 25, the communication portion 26, and the rechargeable battery 27 are housed in the internal space 24a of the housing 24.

The housing 24 according to the present embodiment includes a top wall portion 24b, a bottom wall portion 24c, and a side wall portion 24d. The internal space 24a of the housing 24 is defined by the top wall portion 24b, the bottom wall portion 24c, and the side wall portion 24d. The top wall portion 24b and the bottom wall portion 24c are disposed to face each other in the thickness directions A. An area around the internal space 24a in directions orthogonal to the thickness directions A is defined by the side wall portion 24d. More specifically, the side wall portion 24d according to the present embodiment includes a first side plate portion 24d1 and a second side plate portion 24d2 that each have a flat plate shape and that face each other in the longitudinal directions B and a third side plate portion 24d3 and a fourth side plate portion 24d4 that each have a flat plate shape and that face each other in the width directions C.

In addition, the bottom wall portion 24c according to the present embodiment includes a bottom wall main body portion 24c1 that has a flat plate shape, that is coupled to the side wall portion 24d, and that is formed with an opening at a center and a protrusion portion 24c2 that has a cylindrical shape and that rises from an inner edge portion of the bottom wall main body portion 24c1. A tip of the protrusion portion 24c2 is closed by a tip wall portion 24c3.

The electrodes 21 according to the present embodiment are held by the housing 24 and are exposed to outside of the housing 24. Specifically, the electrodes 21 according to the present embodiment are exposed to outside from the bottom wall portion 24c of the housing 24. More specifically, the electrodes 21 according to the present embodiment protrude from an outer surface of the bottom wall main body portion 24c1 of the bottom wall portion 24c of the housing 24 and are exposed to outside from the bottom wall portion 24c. The protrusion portion 24c2 according to the present embodiment protrudes from the bottom wall main body portion 24c1 more than the electrodes 21. That is, a protrusion amount T1 (see FIG. 8) of the protrusion portion 24c2 from the bottom wall main body portion 24c1 is greater than a protrusion amount T2 (see FIG. 9) of each of the electrodes 21 from the bottom wall main body portion 24c1.

The housing 24 according to the present embodiment includes the detection window portion 24e utilized for detecting living body information by the detection portion 23 housed in the internal space 24a. As described above, the detection portion 23 according to the present embodiment is the optical pulse wave sensor, and the detection portion 23 is configured to detect pulse wave information as the optical transmission/reception portion 23a transmits and receives optical signals through the detection window portion 24e of the housing 24. The detection window portion 24e according to the present embodiment may include, for example, a transparent resin material. The detection window portion 24e according to the present embodiment is formed at the tip wall portion 24c3 of the bottom wall portion 24c of the housing 24.

As illustrated in FIG. 3, the detection window portion 24e according to the present embodiment is disposed in a region surrounded by the four electrodes 21a to 21d in a plan view when the sensor main body 3 is viewed in one of the thickness directions A. However, the detection window portion 24e is not limited in position to the position in the present embodiment.

The control portion 25 executes an operation instruction, for example, provided for each portion of the wearable device 100. The control portion 25 includes a processor such as a central processing unit (CPU) or a microprocessor unit (MPU). More specifically, the control portion 25 according to the present embodiment includes, for example, a storage portion including, for example, a read only memory (ROM) and a random access memory (RAM). The storage portion may store, for example, various programs that the control portion 25 executes. In addition, the wearable device 100 may include a storage portion separately from the control portion 25.

For example, when it has been detected that the pair of detection electrodes 21a and 21b become conductive with the surface of the living body through the conduction portions 41 of the base body 2, the control portion 25 instructs each portion of the wearable device 100 to start measurement operation for living body impedance. Furthermore, for example, when it has been detected that the pair of detection electrodes 21a and 21b become conductive with the surface of the living body through the conduction portions 41 of the base body 2, the control portion 25 according to the present embodiment instructs each portion of the wearable device 100 including the detection portion 23 to allow the detection portion 23 to start measurement of pulse waves.

Furthermore, the control portion 25 may execute processing of calculating an amount of moisture from living body impedance to be measured based on the electrical characteristic of the living body, which the pair of detection electrodes 21a and 21b detect. In addition, the control portion 25 may transmit the calculated amount of moisture to an external device such as a smartphone of the user, a server in a medical institution, or a cloud server, for example, via the communication portion 26. However, the control portion 25 may transmit a measurement value of living body impedance to the external device without executing the processing of calculating an amount of moisture from the living body impedance to be measured based on the electrical characteristic of the living body, which the pair of detection electrodes 21a and 21b detect. That is, the processing of calculating an amount of moisture from living body impedance to be measured based on the electrical characteristic of the living body, which the pair of detection electrodes 21a and 21b detect, may be executed by an external device.

The control portion 25 may execute processing of calculating heartbeats, pulses, and blood pressure, for example, serving as high-order living body information based on pulse waves representing other living body information than the electrical characteristic of the living body, which the optical pulse wave sensor serving as the detection portion 23 detects. In addition, the control portion 25 may transmit the calculated heartbeats, pulses, and blood pressure, for example, serving as the high-order living body information to the external device such as the smartphone of the user, the server in the medical institution, or the cloud server, for example, via the communication portion 26. However, the control portion 25 may transmit the detected living body information itself to the external device without executing the processing of calculating high-order living body information from other living body information than the electrical characteristic of the living body, which the detection portion 23 detects. That is, the processing of calculating high-order living body information based on other living body information than the electrical characteristic of the living body, which the detection portion 23 detects, may be executed by an external device.

The communication portion 26 includes at least one of a wireless communication module and a wired communication module. The wireless communication module is, for example, a communication module compatible with a communication standard such as a wireless local area network (LAN), Bluetooth (registered trademark), or near field communication (NFC). The wired communication module may be, for example, a wired LAN communication module. Thereby, the wearable device 100 is able to perform, for example, wireless communication or wired communication with a communication terminal such as a smartphone or an external device such as a server via the communication portion 26. The communication portion 26 according to the present embodiment includes a wireless communication module including an antenna 26a.

The rechargeable battery 27 is configured to supply electric power to each portion of the wearable device 100.

The sensor main body 3 may further include another component. The sensor main body 3 may further include, for example, a notification portion. For example, the notification portion may be configured, when abnormal living body impedance exceeding a predetermined threshold value is measured, to externally notify, with sound or light, for example, that there is no appropriate attachment to the surface of the living body.

The base body 2 according to the present embodiment includes a back surface 2a that covers an outer surface of the arm X, which serves as the surface of the living body, in a conduction state, and a front surface 2b lying opposite to the back surface 2a. Then, as illustrated in FIGS. 1 - 3 and 6 - 9, the sensor main body 3 according to the present embodiment is configured to be detached from and attached to the base body 2 on a side on which the front surface 2b of the base body 2 is present.

More specifically, the base body 2 according to the present embodiment is the band body 10 that extends in the circumferential directions of the arm X of the user and is configured to be attached to the arm X when wound around the arm X. The band body 10 serving as the base body 2 includes a fitting portion 11 configured to allow the sensor main body 3 to be fitted and a band portion 12 extending to protrude from the fitting portion 11 in the longitudinal directions B of the band body 10.

A housing recess portion (recess) 11a configured to house the sensor main body 3 is formed on the front surface 2b of the base body 2. More specifically, the housing recess portion 11a according to the present embodiment is formed on the front surface 2b at a position of the fitting portion 11 of the base body 2. The housing recess portion 11a is a recess portion having a substantially rectangular shape, and is configured to allow the sensor main body 3 to be engaged. More specifically, an inner surface of the housing recess portion 11a includes a bottom surface 13 and a side surface 14 configured to allow the side wall portion 24d of the housing 24 of the sensor main body 3 to be engaged. More specifically, the side surface 14 of the housing recess portion 11a according to the present embodiment includes a first side surface 14a and a second side surface 14b facing each other in the longitudinal directions B and a third side surface 14c and a fourth side surface 14d facing each other in the width directions C. Although details will be described later, the conduction portions 41 according to the present embodiment are partially exposed on the bottom surface 13 of the housing recess portion 11a. In a state where the sensor main body 3 is housed in the housing recess portion 11a, the electrodes 21 exposed on the bottom wall portion 24c of the housing 24 of the sensor main body 3 come into contact with the conduction portions 41 exposed from the bottom surface 13 of the housing recess portion 11a for conduction.

The band portion 12 includes a first band portion 12a protruding from the fitting portion 11 toward one side in one of the longitudinal directions B and a second band portion 12b protruding from the fitting portion 11 toward another one side in another one of the longitudinal directions B. The first band portion 12a and the second band portion 12b are band-shaped portions having flexibility and configured to be flexibly deformed in the thickness directions A. Therefore, it is possible to wind the first band portion 12a and the second band portion 12b of the band body 10 serving as the base body 2 around the arm X of the user in the circumferential directions of the arm X. One of the first band portion 12a and the second band portion 12b (the first band portion 12a in the present embodiment) has a projection portion 12a1. Another one of the first band portion 12a and the second band portion 12b (the second band portion 12b in the present embodiment) is formed with a plurality of holes 12b1 each allowing the projection portion 12a1 to be inserted. The plurality of holes 12b1 are disposed at intervals in the longitudinal directions B. The projection portion 12a1 is inserted into a corresponding one hole 12b1 among the plurality of holes 12b1 in accordance with how thick the arm X of the user is. Doing so makes it possible to attach the band body 10 serving as the base body 2 to the arm X to allow the conduction portions 41 described later to come into contact with the surface of the living body, regardless of how thick the arm X of the user is.

However, a pair of fasteners of the first band portion 12a and the second band portion 12b are not limited to a pair of the projection portion 12a1 and each of the holes 12b1 described above. The first band portion 12a and the second band portion 12b may form, for example, another pair of fasteners such as a pair of hook-and-loop fasteners. In addition, although the band portion 12 according to the present embodiment includes the first band portion 12a and the second band portion 12b, the present disclosure is not limited to this configuration. The band portion 12 may include only one band-shaped portion. In such a case, the band portion 12 may protrude from one end portion in the longitudinal directions B of the fitting portion 11, and, in the state of being wound around the arm X of the user, may be locked, at its tip portion, to another end portion in the longitudinal directions B of the fitting portion 11.

As described above, the base body 2 includes the conduction portions 41. The conduction portions 41 according to the present embodiment will now be described herein in detail.

The base body 2 according to the present embodiment includes the four conduction portions 41. The four conduction portions 41 are configured to come into contact with and configured to be conductive with the four electrodes 21a to 21d of the sensor main body 3, respectively. Specifically, the base body 2 serving as the attachment portion 1 according to the present embodiment includes a pair of the conduction portions 41 that come into contact with and become conductive with the pair of detection electrodes 21a and 21b of the sensor main body 3, respectively, and a pair of the conduction portions 41 that come into contact with and become conductive with the pair of auxiliary electrodes 21c and 21d of the sensor main body 3, respectively. More specifically, the four conduction portions 41 according to the present embodiment include a first conduction portion 41a configured to come into contact with one of the pair of detection electrodes 21a and 21b, that is, the detection electrode 21a, a second conduction portion 41b configured to come into contact with another one of the pair of detection electrodes 21a and 21b, that is, the detection electrode 21b, a third conduction portion 41c configured to come into contact with one of the pair of auxiliary electrodes 21c and 21d, that is, the auxiliary electrode 21c, and a fourth conduction portion 41d configured to come into contact with another one of the pair of auxiliary electrodes 21c and 21d, that is, the auxiliary electrode 21d. The sensor main body 3 is fitted to the base body 2 to allow any of the four electrodes 21a to 21d to be in a state of being in contact with a corresponding one of the first to fourth conduction portions 41a to 41d. The first to fourth conduction portions 41a to 41d will be hereinafter simply referred to as the “conduction portions 41” unless otherwise distinguished.

The conduction portions 41 each include a front surface exposure portion 42 exposed on the front surface 2b of the base body 2 and a back surface exposure portion 43 exposed on the back surface 2a of the base body 2. The front surface exposure portions 42 are configured to come into contact with the electrodes 21 of the sensor main body 3 fitted to the base body 2, respectively. The back surface exposure portions 43 are configured to come into contact with the surface of the living body in a state where the base body 2 is attached to the surface of the living body, respectively.

The front surface exposure portions 42 of the conduction portions 41 according to the present embodiment are exposed on a front surface of the fitting portion 11 on the front surface 2b of the base body 2. More specifically, the front surface exposure portions 42 of the conduction portions 41 according to the present embodiment are exposed on the bottom surface 13 of the housing recess portion 11a formed on the front surface of the fitting portion 11. Therefore, when the sensor main body 3 is housed in the housing recess portion 11a, the electrodes 21 exposed on an outer surface of the bottom wall portion 24c of the housing 24, which serves as a back surface of the sensor main body 3, come into contact with the front surface exposure portions 42 of the conduction portions 41, respectively. In other words, the sensor main body 3 is inserted into the housing recess portion 11a to a position at which the electrodes 21 come into contact with the conduction portions 41, respectively.

The front surface exposure portions 42 and the back surface exposure portions 43 of the conduction portions 41 are disposed at positions overlapping each other in the longitudinal directions B representing the extending directions of the band body 10 serving as the base body 2. That is, the back surface exposure portions 43 of the conduction portions 41 according to the present embodiment are exposed on a back surface of the fitting portion 11 on the back surface 2a of the base body 2. In the use state of the wearable device 100 illustrated in FIGS. 4 and 5, the fitting portion 11 is disposed on an outer surface on a back side of a hand on the outer surface of the arm X of the user. Therefore, in the use state of the wearable device 100 illustrated in FIGS. 4 and 5, the back surface exposure portions 43 of the conduction portions 41 come into contact with the outer surface of the back side of the hand on the outer surface of the arm X of the user. However, it is also possible to use the wearable device 100 according to the present embodiment to allow the fitting portion 11 to be disposed on the outer surface on a palm side of the hand on the outer surface of the arm X of the user. In such a case, the back surface exposure portions 43 of the conduction portions 41 come into contact with the outer surface on the palm side of the hand on the outer surface of the arm X of the user, respectively.

In addition, it is preferable that the back surface exposure portions 43 of the conduction portions 41 protrude on the back surface 2a of the base body 2. Doing so allows, in a state where the base body 2 is attached to the surface of the living body (see FIGS. 4 and 5), the back surface exposure portions 43 to easily slightly push and abut the surface of the living body. Doing so makes it possible to stabilize the contact state between the conduction portions 41 and the surface of the living body.

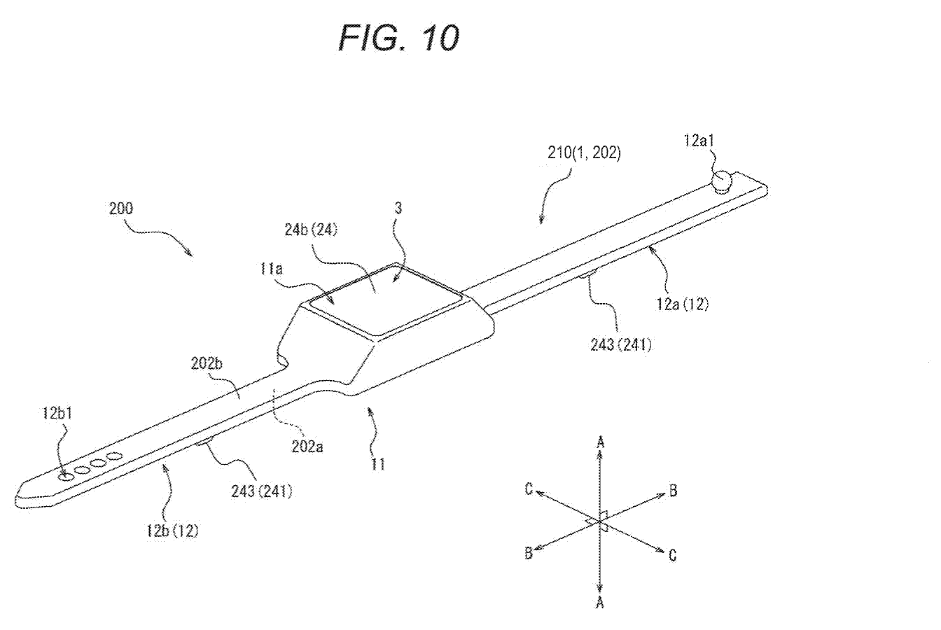

As described above, although the front surface exposure portions 42 and the back surface exposure portions 43 of the conduction portions 41 according to the present embodiment are disposed at the positions overlapping each other in the longitudinal directions B representing the extending directions of the band body 10 serving as the base body 2, the present disclosure is not limited to this configuration. The front surface exposure portions 42 and the back surface exposure portions 43 of the conduction portions 41 may be disposed at positions not overlapping each other in the longitudinal directions B representing the extending directions of the band body 10. Such a configuration as described above will be described later in detail (see FIGS. 10 - 14).

As described above, the wearable device 100 according to the present embodiment is configured to make the electrodes 21 of the sensor main body 3 and the surface of the living body conductive with each other via the conduction portions 41 of the base body 2, respectively. More specifically, the wearable device 100 according to the present embodiment is configured to make the detection electrode 21a and the surface of the living body conductive with each other via the first conduction portion 41a of the base body 2. In addition, the wearable device 100 according to the present embodiment is configured to make the detection electrode 21b and the surface of the living body conductive with each other via the second conduction portion 41b of the base body 2. Furthermore, the wearable device 100 according to the present embodiment is configured to make the auxiliary electrode 21c and the surface of the living body conductive with each other via the third conduction portion 41c of the base body 2. Furthermore, the wearable device 100 according to the present embodiment is also configured to make the auxiliary electrode 21d and the surface of the living body conductive with each other via the fourth conduction portion 41d of the base body 2.

As described above, the four electrodes 21a to 21d of the sensor main body 3 according to the present embodiment do not come into direct contact with the surface of the living body in the conduction state. In addition, the sensor main body 3 according to the present embodiment includes no electrodes other than the four electrodes 21a to 21d. That is, the sensor main body 3 according to the present embodiment includes no electrodes that are configured to directly come into contact with the surface of the living body in the conduction state.

A constituent material from which the conduction portions 41 may be fabricated is not particularly limited as long as a material having conductivity is used. The conduction portions 41 may include, for example, metal such as copper or aluminum. However, it is preferable that the conduction portions 41 include conductive rubber that is flexible and has a high skin contact property. Examples of the conductive rubber include conductive silicone rubber and conductive styrene-based rubber. In addition, other constituent materials than that of the conduction portions 41 in the base body 2 according to the present embodiment may be, for example, resin having no conductivity.

In addition, the opening portion 50 is formed on the base body 2. The opening portion 50 penetrates from a side on which the front surface 2b is present to a side on which the back surface 2a is present. More specifically, the opening portion 50 according to the present embodiment is a through hole penetrating from the front surface 2b to the back surface 2a of the base body 2 in the thickness directions A. The detection portion 23 of the sensor main body 3 according to the present embodiment is configured to detect pulse wave information and blood pressure information serving as living body information through the opening portion 50 of the base body 2 in the conduction state. Since there is the opening portion 50 as described above, the base body 2 does not hinder detection of living body information by the detection portion 23 of the sensor main body 3. That is, the base body 2 being interposed makes it possible to suppress a decrease in detection accuracy of living body information by the detection portion 23.

As illustrated in FIG. 8, the sensor main body 3 according to the present embodiment is inserted into the opening portion 50 from the side on which the front surface 2b of the base body 2 is present in the conduction state. More specifically, the protrusion portion 24c2 of the bottom wall portion 24c of the housing 24 of the sensor main body 3 according to the present embodiment is inserted into the opening portion 50 from the side on which the front surface 2b of the base body 2 is present in the conduction state. Therefore, it is possible to bring the detection window portion 24e formed on the tip wall portion 24c3 disposed at the tip of the protrusion portion 24c2 close to the surface of the living body. That is, it is possible to reduce a gap between the detection window portion 24e formed on the tip wall portion 24c3 and the surface of the living body, making it possible to suppress a decrease in detection accuracy by the detection portion 23, which may occur due to that such a gap is present.

Furthermore, the sensor main body 3 according to the present embodiment protrudes from the back surface 2a of the base body 2 through the opening portion 50 of the base body 2 in the conduction state. More specifically, the protrusion portion 24c2 of the bottom wall portion 24c is inserted into the opening portion 50 from the side on which the front surface 2b of the base body 2 is present to allow the tip wall portion 24c3 of the bottom wall portion 24c of the housing 24 of the sensor main body 3 according to the present embodiment to protrude from the back surface 2a of the base body 2 through the opening portion 50 of the base body 2. Doing so makes it possible to allow the detection window portion 24e formed on the tip wall portion 24c3 to come into contact with the surface of the living body. That is, it is possible to eliminate a gap between the detection window portion 24e formed on the tip wall portion 24c3 and the surface of the living body, making it possible to further suppress a decrease in detection accuracy by the detection portion 23.

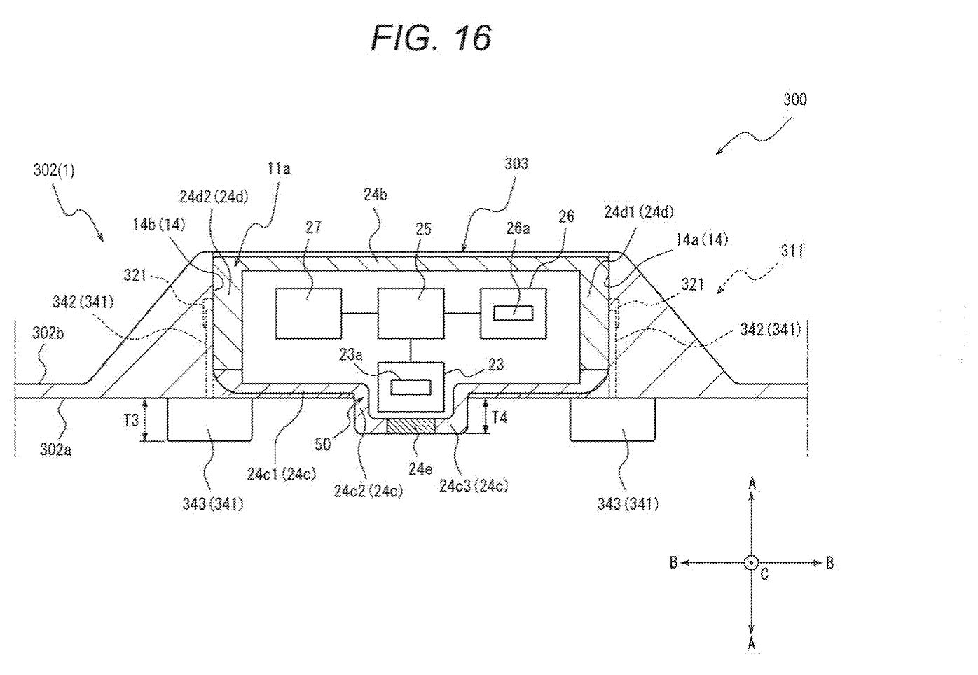

As described above, it is preferable that the back surface exposure portions 43 of the conduction portions 41 protrude from the back surface 2a of the base body 2. In this case, it is preferable that a protrusion amount of each of the back surface exposure portions 43 of the conduction portions 41 from the back surface 2a of the base body 2 is greater than a protrusion amount of the sensor main body 3 from the back surface 2a of the base body 2, as described above. Doing so makes it possible to suppress difficulties in allowing the conduction portions 41 and the surface of the living body to come into contact with each other, since the sensor main body 3 protrudes through the opening portion 50. Such a relationship in protrusion amount as described above is illustrated in a wearable device 300 to be described later (see a protrusion amount T3 of each of back surface exposure portions 343 of conduction portions 341 illustrated in FIG. 16 and a protrusion amount T4 of the protrusion portion 24c2 illustrated in FIG. 16).

Although, in the base body 2 according to the present embodiment, the through hole serving as the opening portion 50 is formed, the opening portion 50 is not limited to the through hole. The opening portion 50 may be, for example, a recess portion having a cut-away shape, which penetrates in the thickness directions A of the base body 2 and opens to an outer edge in the width directions C of the base body 2.

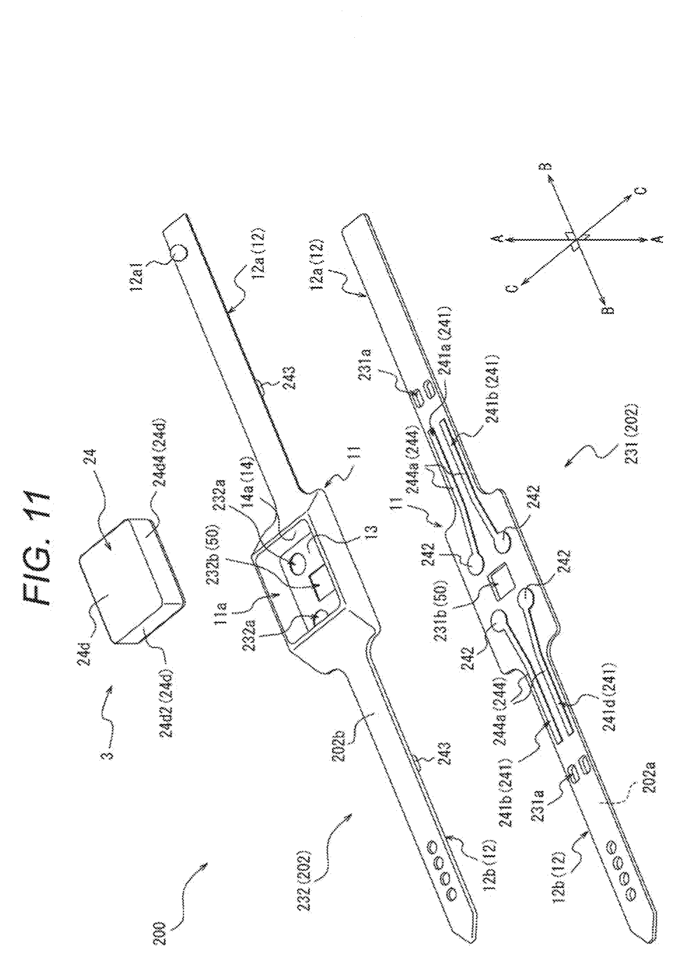

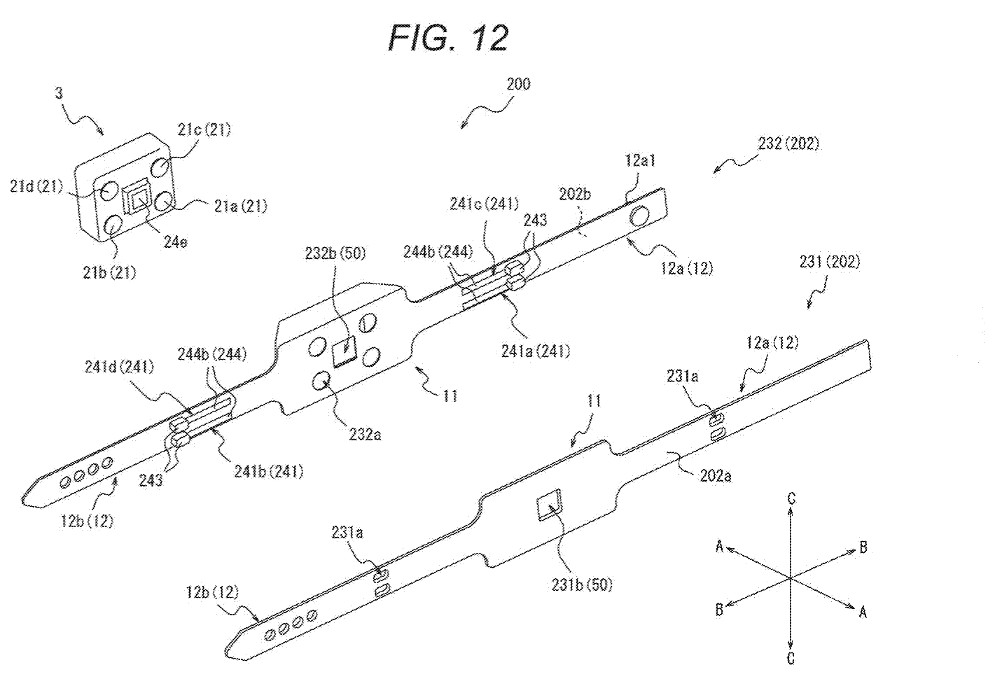

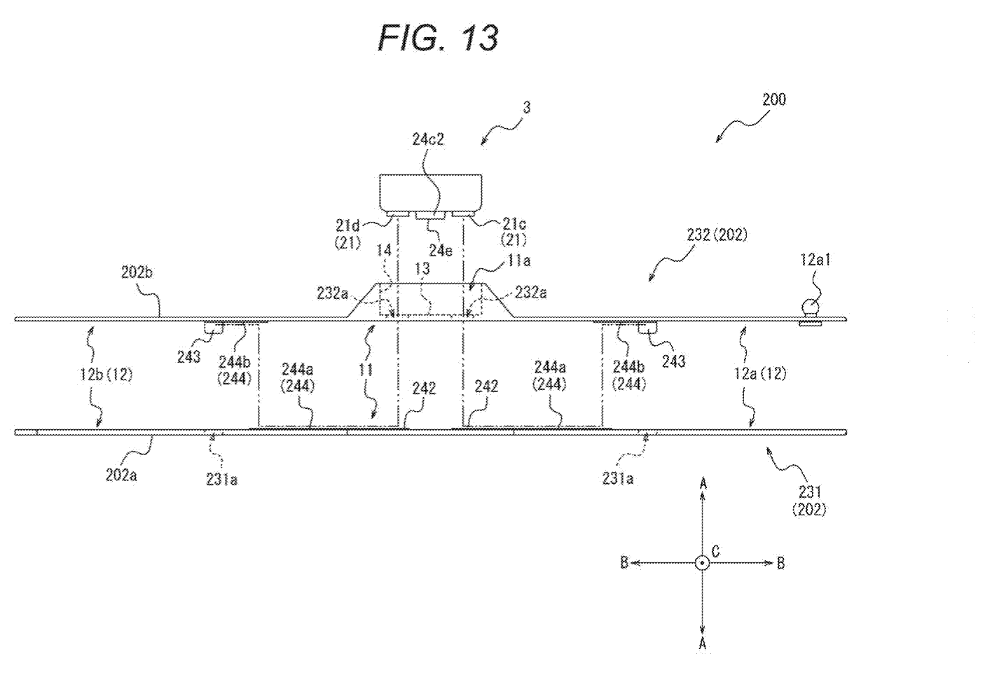

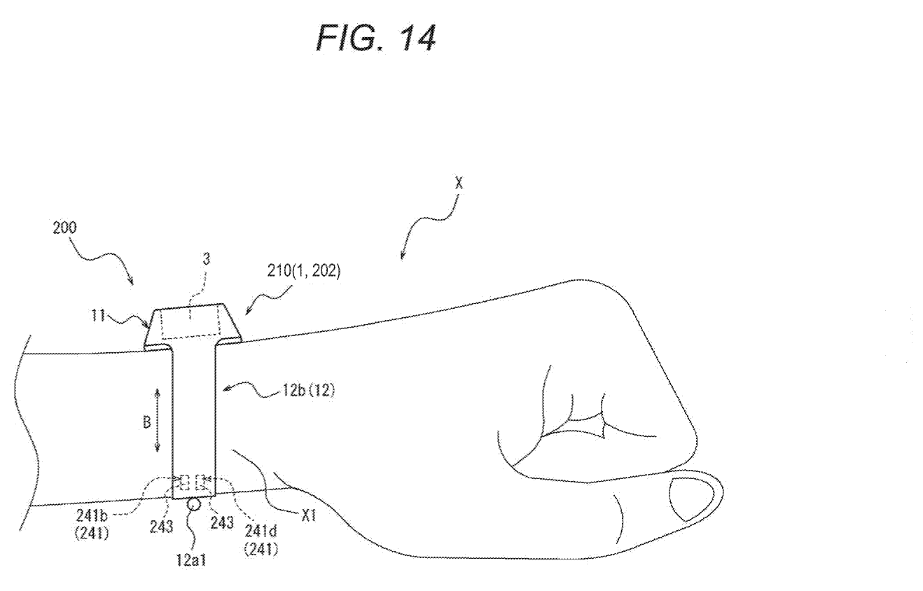

Next, a wearable device 200 serving as another embodiment of the wearable device according to the present disclosure will now be described herein by way of example with reference to FIGS. 10- 14. FIG. 10 is a perspective view when the wearable device 200 is viewed from a side on which its front surface is present. As illustrated in FIG. 10, the wearable device 200 according to the present embodiment includes a base body 202 serving as the attachment portion 1 and the sensor main body 3. FIG. 10 illustrates a state where the sensor main body 3 is fitted to the base body 202. FIGS. 11 and 12 are exploded perspective views of the wearable device 200, illustrating that the base body 202 and the sensor main body 3 of the wearable device 200 are separated from each other. In addition, FIGS. 11 and 12 illustrate a state where the base body 202 is further disassembled into a base main body 231 and a base cover 232. FIG. 11 is the exploded perspective view when the wearable device 200 in a state where the base main body 231 of the base body 202, the base cover 232 of the base body 202, and the sensor main body 3 are separated from each other is viewed from the side on which the front surface is present. FIG. 12 is the exploded perspective view when the wearable device 200 in a state where the base main body 231 of the base body 202, the base cover 232 of the base body 202, and the sensor main body 3 are separated from each other is viewed from a side on which its back surface is present. FIG. 13 is a side view of the base main body 231 of the base body 202, the base cover 232 of the base body 202, and the sensor main body 3 in a separated state. In FIG. 13, a conduction path from the electrodes 21 of the sensor main body 3 to back surface exposure portions 243 of conduction portions 241 of the base body 202 is indicated by a two-dot chain line. FIG. 14 is a view illustrating the use state of the wearable device 200 illustrated in FIG. 10.

Since the sensor main body 3 is identical or similar in configuration to that in the wearable device 100 described above, its description will be omitted herein.

The base body 202 according to the present embodiment is a band body 210 configured to be attached to the arm X when wound around to extend in the circumferential directions of the arm X of the user. The band body 210 serving as the base body 202 according to the present embodiment is mainly different in the configurations of the conduction portions 241 from the band body 10 (see FIG. 1, for example) according to the embodiment described above. More specifically, while, in the band body 10 (see FIG. 1, for example) according to the embodiment described above, the front surface exposure portions 42 and the back surface exposure portions 43 of the conduction portions 41 are disposed at the positions overlapping each other in the longitudinal directions B representing the extending directions of the band body 10, in the band body 210 according to the present embodiment, as illustrated in FIG. 13, front surface exposure portions 242 and the back surface exposure portions 243 of the conduction portions 241 do not overlap each other in the longitudinal directions B representing the extending directions of the band body 10, and are disposed at different positions in the longitudinal directions B.

Therefore, as illustrated in FIG. 14, it is possible to allow the back surface exposure portions 243 of the conduction portions 241 to come into contact with the surface of the living body at positions different from a position of the fitting portion 11 in the longitudinal directions B, that is, at a position of the band portion 12. There may be cases where hair is present on the outer surface on the back side of the hand on the outer surface of the arm X of the user. Therefore, there may be cases where hair is interposed between one of the back surface exposure portions 243 of the conduction portions 241 and the skin, and the hair may decrease detection accuracy of the electrical characteristic of the living body, which is to be detected through the conduction portions 241. In addition, the outer surface on the back side of the hand on the outer surface of the arm X of the user generally has less fat than the outer surface on the palm side of the hand, and there is a possibility that the contact state with the back surface exposure portions 243 of the conduction portions 241 is not stable. Therefore, it is preferable that the back surface exposure portions 243 of the conduction portions 241 are allowed to come into contact with the outer surface on the palm side of the hand rather than the outer surface on the back side of the hand on the outer surface of the arm X of the user. In the present embodiment, the back surface exposure portions 243 of the conduction portions 241 are exposed on a back surface of the band portion 12, on a back surface 202a of the base body 202. Thereby, it is possible to allow the back surface exposure portions 243 of the conduction portions 241 to come into contact with the outer surface on the palm side of the hand of the arm X of the user. As a result, it is possible to suppress a decrease in detection accuracy of the electrical characteristic of the living body, which is to be detected through the conduction portions 241 and to stabilize the contact state between the conduction portions 241 and the surface of the living body.

The base body 202 according to the present embodiment will now be described herein in detail.

As described above, the base body 202 according to the present embodiment includes the base main body 231 and the base cover 232.

A back surface of the base main body 231 serves as the back surface 202a of the base body 202. The base cover 232 is stacked on a front surface of the base main body 231. A front surface of the base cover 232 serves as a front surface 202b of the base body 202. As described above, the base body 202 according to the present embodiment is formed as the base main body 231 and the base cover 232 are stacked with each other.

As illustrated in FIG. 10, the sensor main body 3 according to the present embodiment is configured to be detached from and attached to the base body 202 on a side on which the front surface 202b of the base body 202 is present.

More specifically, the band body 210 serving as the base body 202 according to the present embodiment includes the fitting portion 11 configured to allow the sensor main body 3 to be fitted and the band portion 12 extending to protrude from the fitting portion 11 in the longitudinal directions B of the band body 210.

The housing recess portion 11a configured to house the sensor main body 3 is formed on the front surface 202b of the base body 202. More specifically, the housing recess portion 11a according to the present embodiment is formed on the front surface 202b at a position of the fitting portion 11 of the base body 202. Since a shape of the housing recess portion 11a is identical or similar in configuration to that in the wearable device 100 described above, its description will not be repeated here. Although details will be described later, the front surface exposure portions 242 of the conduction portions 241 according to the present embodiment are exposed on the bottom surface 13 of the housing recess portion 11a. In a state where the sensor main body 3 is housed in the housing recess portion 11a, the electrodes 21 exposed from the bottom wall portion 24c of the housing 24 of the sensor main body 3 come into contact with the front surface exposure portions 242 of the conduction portions 241, which are exposed on the bottom surface 13 of the housing recess portion 11a for conduction.

The band portion 12 includes the first band portion 12a protruding from the fitting portion 11 toward one side in one of the longitudinal directions B and the second band portion 12b protruding from the fitting portion 11 toward another one side in another one of the longitudinal directions B. The first band portion 12a and the second band portion 12b are band-shaped portions having flexibility and configured to be flexibly deformed in the thickness directions A. Therefore, it is possible to wind the first band portion 12a and the second band portion 12b of the band body 210 serving as the base body 202 around the arm X of the user in the circumferential directions of the arm X. One of the first band portion 12a and the second band portion 12b (the first band portion 12a in the present embodiment) has the projection portion 12a1. Another one of the first band portion 12a and the second band portion 12b (the second band portion 12b in the present embodiment) is formed with the plurality of holes 12b1 each allowing the projection portion 12a1 to be inserted. The plurality of holes 12b1 are disposed at intervals in the longitudinal directions B. The projection portion 12a1 is inserted into a corresponding one hole 12b1 among the plurality of holes 12b1 in accordance with how thick the arm X of the user is. Doing so makes it possible to attach the band body 10 serving as the base body 2 to the arm X to allow the conduction portions 241 described later to come into contact with the surface of the living body, regardless of how thick the arm X of the user is.

However, the pair of fasteners of the first band portion 12a and the second band portion 12b are not limited to the pair of the projection portion 12a1 and each of the holes 12b1 described above. The first band portion 12a and the second band portion 12b may form, for example, another pair of fasteners such as a pair of hook-and-loop fasteners. In addition, although the band portion 12 according to the present embodiment includes the first band portion 12a and the second band portion 12b, the present disclosure is not limited to this configuration. The band portion 12 may include only one band-shaped portion. In such a case, the band portion 12 may protrude from one end portion in the longitudinal directions B of the fitting portion 11, and, in the state of being wound around the arm X of the user, may be locked, at its tip portion, to another end portion in the longitudinal directions B of the fitting portion 11.

Next, the conduction portions 241 of the base body 202 according to the present embodiment will now be described herein in detail.

The base body 202 according to the present embodiment includes the four conduction portions 241. The four conduction portions 241 are configured to come into contact with and configured to be conductive with the four electrodes 21ato 21d of the sensor main body 3, respectively. Specifically, the four conduction portions 241 according to the present embodiment include a first conduction portion 241a configured to come into contact with the detection electrode 21a, a second conduction portion 241b configured to come into contact with the detection electrode 21b, a third conduction portion 241c configured to come into contact with the auxiliary electrode 21c, and a fourth conduction portion 241d configured to come into contact with the auxiliary electrode 21d. The sensor main body 3 is fitted to the base body 2 to allow any of the four electrodes 21ato 21d to be in a state of being in contact with a corresponding one of the first to fourth conduction portions 241a to 241d. The first to fourth conduction portions 241a to 241d will be hereinafter simply referred to as the “conduction portions 241” unless otherwise distinguished.

The conduction portions 241 each include the front surface exposure portion 242 exposed on the front surface 202b of the base body 202 and the back surface exposure portion 243 exposed on the back surface 202a of the base body 202. The front surface exposure portions 242 are configured to come into contact with the electrodes 21 of the sensor main body 3 fitted to the base body 202, respectively. The back surface exposure portions 243 are configured to come into contact with the surface of the living body in a state where the base body 202 is attached to the surface of the living body, respectively.

The front surface exposure portions 242 of the conduction portions 241 according to the present embodiment are exposed on the front surface of the fitting portion 11 on the front surface 202b of the base body 202. More specifically, the front surface exposure portions 242 of the conduction portions 241 according to the present embodiment are exposed on the bottom surface 13 of the housing recess portion 11a formed on the front surface of the fitting portion 11. Therefore, when the sensor main body 3 is housed in the housing recess portion 11a, the electrodes 21 exposed on the outer surface of the bottom wall portion 24c of the housing 24, which serves as the back surface of the sensor main body 3, come into contact with the front surface exposure portions 242 of the conduction portions 241, respectively. In other words, the sensor main body 3 is inserted into the housing recess portion 11a to a position at which the electrodes 21 come into contact with the conduction portions 241, respectively.

As described above, in the present embodiment, the front surface exposure portions 242 and the back surface exposure portions 243 of the conduction portions 241 are disposed at the positions not overlapping each other in the longitudinal directions B representing the extending directions of the band body 210 serving as the base body 202. More specifically, the back surface exposure portions 243 of the conduction portions 241 according to the present embodiment are exposed on the back surface of the band portion 12 instead of the back surface of the fitting portion 11 on the back surface 202a of the base body 202.

More specifically, the conduction portions 241 according to the present embodiment include wiring portions 244 that electrically couple the front surface exposure portions 242 and the back surface exposure portions 243 to each other and are embedded in the band body 210 without being exposed to outside. As there are the wiring portions 244 described above, the front surface exposure portions 242 and the back surface exposure portions 243 of the conduction portions 241 are disposed at different positions in the longitudinal directions B. In addition, since the wiring portions 244 are embedded in the band body 210, it is possible to suppress the wiring portions 244 from being damaged or corroded. The wiring portions 244 may be allowed to undergo insulation treatment on their outer surfaces and may be disposed on the back surface 202a or the front surface 202b of the band body 210. However, from a viewpoint of suppressing damage, it is preferable that the wiring portions 244 are embedded in the band body 210, identical or similar to the wiring portions 244 according to the present embodiment.

The wiring portions 244 according to the present embodiment include first conduction paths 244a that are coupled to the front surface exposure portions 242 and extend in the longitudinal directions B on the front surface of the base body 231. In addition, the wiring portions 244 according to the present embodiment include second conduction paths 244b that are coupled to the back surface exposure portions 243 and extend in the longitudinal directions B on the back surface of the base cover 232. The first conduction paths 244a and the second conduction paths 244b are configured to be in contact with and configured to be conductive with each other as the base main body 231 and the base cover 232 are stacked with each other. However, the wiring portions 244 are not limited to be in the configuration including the first conduction paths 244a and the second conduction paths 244b described above.

In addition, in the present embodiment, the front surface exposure portions 242 of the conduction portions 241 are fixed on the front surface of the base main body 231. The front surface exposure portions 242 of the conduction portions 241 are configured to be exposed from the bottom surface 13 of the housing recess portion 11a via through holes 232a formed on the base cover 232 as the base main body 231 and the base cover 232 are stacked with each other. However, the front surface exposure portions 242 of the conduction portions 241 are not limited to be in the configuration of being fixed on the front surface of the base main body 231. The front surface exposure portions 242 of the conduction portions 241 may be fixed on the side on which the base cover 232 is present, for example.

Furthermore, in the present embodiment, the back surface exposure portions 243 of the conduction portions 241 are fixed on the back surface of the base cover 232. The back surface exposure portions 243 of the conduction portions 241 are configured to be exposed on the back surface 202a of the base body 202 via through holes 231a formed on the base main body 231 as the base main body 231 and the base cover 232 are stacked with each other. However, the back surface exposure portions 243 of the conduction portions 241 are not limited to be in the configuration of being fixed on the back surface of the base cover 232. The back surface exposure portions 243 of the conduction portions 241 may be fixed on the side on which the base main body 231 is present, for example.

A constituent material from which the conduction portions 241 may be fabricated is not particularly limited as long as a material having conductivity is used. The conduction portions 241 may include, for example, metal such as copper or aluminum. However, in the conduction portions 241, it is preferable that the back surface exposure portions 243 that come into contact with the surface of the living body include conductive rubber that is flexible and has a high skin contact property. Examples of the conductive rubber include conductive silicone rubber and conductive styrene-based rubber. In addition, other constituent materials than that of the conduction portions 241 in the base body 202 according to the present embodiment may be, for example, resin having no conductivity.

In addition, the opening portion 50 is formed on the base body 202. The opening portion 50 penetrates from the side on which the front surface 202b is present to the side on which the back surface 202a is present. More specifically, the opening portion 50 according to the present embodiment is a through hole penetrating from the front surface 202b to the back surface 202a of the base body 202 in the thickness directions A. The through hole serving as the opening portion 50 according to the present embodiment is formed as a first opening portion 231b formed on the base main body 231 and a second opening portion 232b formed on the base cover 232 are allowed to communicate with each other in the thickness directions A. In other words, the base main body 231 and the base cover 232 according to the present embodiment are stacked with each other to allow the first opening portion 231b and the second opening portion 232b to communicate with each other in the thickness directions A. Since a relationship between the sensor main body 3 and the opening portion 50 is identical or similar to that in the wearable device 100 (see FIGS. 1 - 9) described above, its description will be omitted in here.

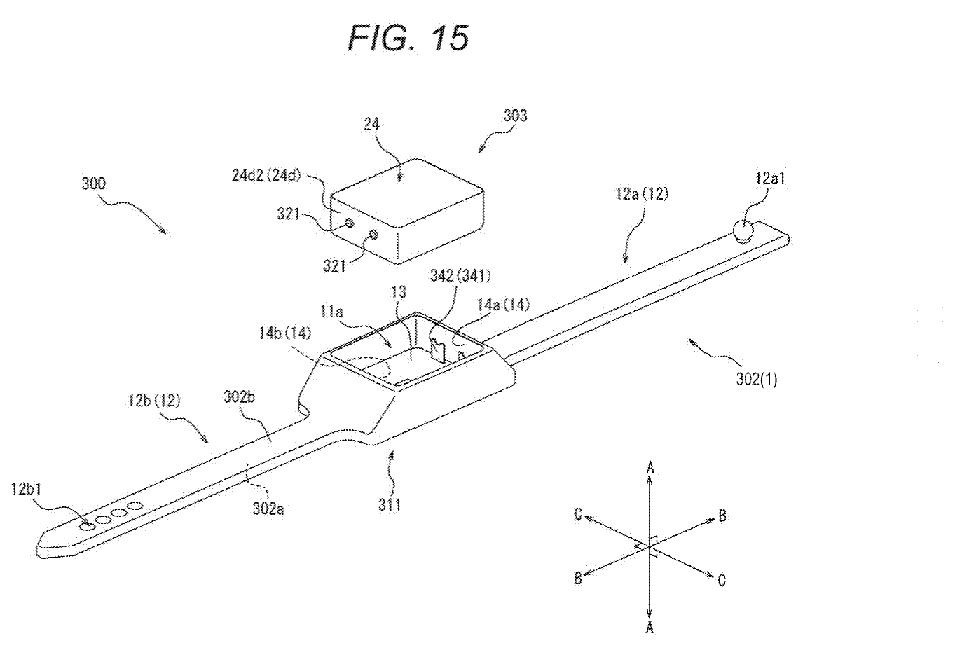

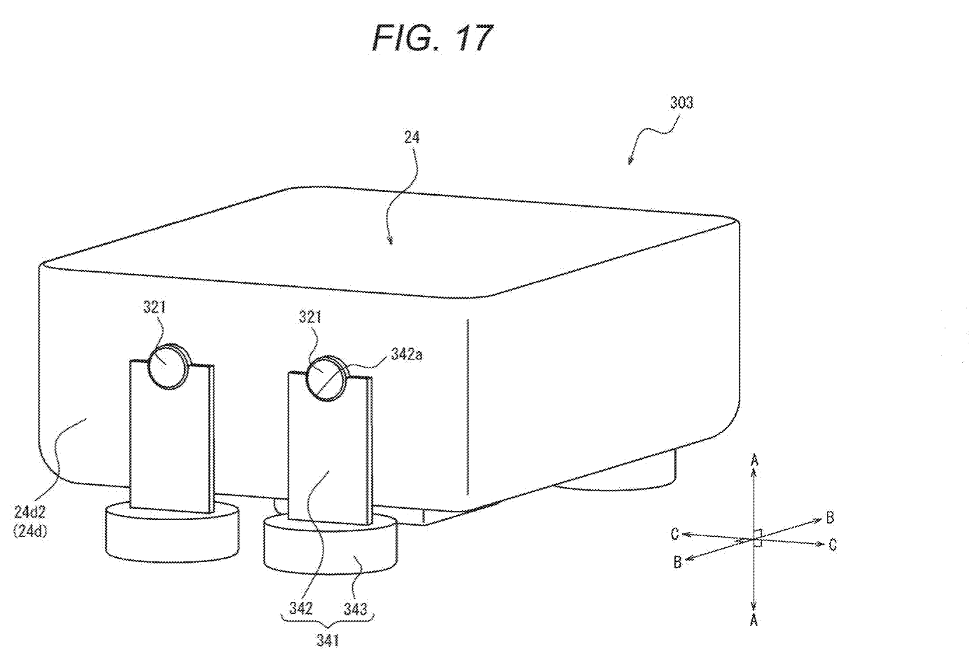

Next, the wearable device 300 serving as still another embodiment of the wearable device according to the present disclosure will now be described herein by way of example with reference to FIGS. 15 -17. FIG. 15 is an exploded perspective view of the wearable device 300. As illustrated in FIG. 15, the wearable device 300 includes a base body 302 serving as the attachment portion 1 and a sensor main body 303 configured to be detached from and attached to the base body 302. FIG. 16 is a cross-sectional view of a fitting portion 311 of the wearable device 300. FIG. 17 is a view illustrating a contact state between the conduction portions 341 of the base body 302 and electrodes 321 of the sensor main body 303.

Although, in the wearable device 300 according to the present embodiment, a configuration of the conduction portions 341 of the base body 302 and exposure positions of the electrodes 321 of the sensor main body 303 differ, compared with the wearable device 100 (see FIGS. 1 -9) described above, other configurations remain identical. Only such differences as described above will be described in here, and a description of features and aspects identical to those in the wearable device 100 (see FIGS. 1 - 9) will not be repeated here.

Front surface exposure portions 342 of the conduction portions 341 of the base body 302 according to the present embodiment are exposed on the side surface 14 of the housing recess portion 11a on the front surface 302b of the base body 302. As illustrated in FIG. 17, engagement recess portions 342a into which the electrodes 321 of the sensor main body 303 are inserted are formed on the front surface exposure portions 342 according to the present embodiment. In addition, the electrodes 321 of the sensor main body 303 according to the present embodiment are held by the housing 24 and are exposed to outside of the housing 24. Specifically, the electrodes 321 according to the present embodiment are exposed to outside from the side wall portion 24d of the housing 24. More specifically, the electrodes 321 according to the present embodiment are exposed to outside from the side wall portion 24d of the housing 24 to protrude from respective outer surfaces of the first side plate portion 24d1 and the second side plate portion 24d2 that each have a flat plate shape and that face each other in the longitudinal directions B. Then, as the sensor main body 303 is housed in the housing recess portion 11a of the base body 302, the electrodes 321 according to the present embodiment enter the engagement recess portions 342a formed on the front surface exposure portions 342 of the conduction portions 341, and come into contact with and become conductive with the front surface exposure portions 342, respectively.

As described above, the front surface exposure portions 342 of the conduction portions 341 of the base body 302 may be configured to be exposed on the side surface 14 of the housing recess portion 11a. The housing recess portion 11a is formed on a front surface 302b of the base body 302, and the bottom surface 13 and the side surface 14 representing the inner surface of the housing recess portion 11a also serve as parts of the front surface 302b of the base body 302.

In addition, although the back surface exposure portions 343 of the conduction portions 341 according to the present embodiment are disposed at the positions overlapping the front surface exposure portions 342 in the longitudinal directions B, respectively, the present disclosure is not limited to this configuration. That is, the back surface exposure portions 343 may be exposed on a back surface 302a of the base body 302 at positions not overlapping the front surface exposure portions 342 in the longitudinal directions B, respectively. Therefore, the back surface exposure portions 343 may be exposed on the back surface of the band portion 12, for example. In such a case, for example, the wiring portions 244 (see FIG. 11, for example) of the conduction portions 241 of the wearable device 200 (see FIGS. 10 - 14) described above may be used to electrically couple the front surface exposure portions 342 and the back surface exposure portions 343 disposed at the different positions in the longitudinal directions B to each other.

The wearable device according to the present disclosure is not limited to have the specific configurations illustrated in the embodiments described above, and it is possible to make various changes, modifications, and combinations without departing from the scope of the claims. Although, in the wearable devices illustrated in the embodiments described above, the configuration in which the sensor main body includes the four electrodes and the base body includes the four conduction portions respectively corresponding to the four electrodes has been exemplified and described, the present disclosure is not limited to this configuration. The electrodes of the sensor main body and the conduction portions of the base body may be each, for example, less than four or five or more in number. In addition, although, in the wearable devices illustrated in the embodiments described above, the optical pulse wave sensor has been described as an example of the detection portion of the sensor main body, another sensor configured to detect an electromagnetic wave or a sound wave from a living body may be used. Examples of such another sensor described above include, for example, a microwave sensor and a sound sensor configured to detect various sounds that a living body emits.

In addition, although the wearable devices illustrated in the embodiments described above each have the configuration including, as the attachment portion, a base body that is to be attached to the surface of the living body and that is to be interposed between the surface of the living body and the sensor main body, the attachment portion is not limited to the base body. For example, the attachment portion may be configured to be attached on the surface of the living body without being interposed between the surface of the living body and the sensor main body. The attachment portion described above may have a configuration in which, for example, no fitting portion formed with a housing recess portion configured to house the sensor main body is included, and a first band portion and a second band portion separated from each other are directly fitted on a side of the sensor main body. In this case, the conduction portions respectively have contacts electrically coupled to the electrodes of the sensor main body at positions fitted to the side of the sensor main body on the first band portion and the second band portion, and respectively have the back surface exposure portions configured to come into contact with the surface of the living body on respective back surfaces of the first band portion and the second band portion. In addition, in this case, the attachment portion may have a configuration of not solely attached to the surface of the living body, and of being configured to be attached to the surface of the living body upon establishment of a state where the sensor main body is fitted. Including such a configuration as described above, the attachment portion is configured to be attached to the surface of the living body.

The detailed description above describes embodiments of a wearable device representing examples of the new wearable device disclosed here. The invention is not limited, however, to the precise embodiments and variations described. Various changes, modifications and equivalents can be effected by one skilled in the art without departing from the spirit and scope of the invention as defined in the accompanying claims. It is expressly intended that all such changes, modifications and equivalents that fall within the scope of the claims are embraced by the claims.

What is claimed is:

1. A wearable device comprising:
a sensor main body including an electrode configured to detect an electrical characteristic of a living body and a detection portion configured to detect living body information other than the electrical characteristic of the living body;
an attachment portion configured to be attached to and detached from the sensor main body and configured to be attached to a surface of the living body;
the attachment portion including a conduction portion that makes the electrode of the sensor main body and the surface of the living body conductive with each other when the electrode of the sensor main body and the surface of the living body come into contact with each other; and
the attachment portion being configured to not act as a blockage between the detection portion of the sensor main body and the surface of the living body in a conduction state in which the attachment portion fitted with the sensor main body is attached to the surface of the living body and the electrode of the sensor main body and the surface of the living body are conductive with each other via the conduction portion of the attachment portion.

2. The wearable device according to claim 1, wherein
the attachment portion includes a back surface is configured to cover the surface of the living body in the conduction state and a front surface lying opposite to the back surface,
the sensor main body is configured to be detached from and attached to the attachment portion on a side on which the front surface of the attachment portion is present,
an opening portion penetrating from the side on which the front surface is present to a side on which the back surface is present is formed on the attachment portion, and
the detection portion of the sensor main body is configured to detect the living body information through the opening portion of the attachment portion in the conduction state.

3. The wearable device according to claim 2, wherein the sensor main body is inserted into the opening portion from the side on which the front surface of the attachment portion is present in the conduction state.

4. The wearable device according to claim 3, wherein the sensor main body protrudes from the back surface of the attachment portion through the opening portion of the attachment portion in the conduction state.

5. The wearable device according to claim 2, wherein the conduction portion of the attachment portion includes:
a front surface exposure portion that is exposed on the front surface and is configured to come into contact with the electrode of the sensor main body; and
a back surface exposure portion that is exposed on the back surface and is configured to come into contact with the surface of the living body.

6. The wearable device according to claim 5, wherein
a housing recess portion configured to house the sensor main body is formed on the front surface of the attachment portion, and
the front surface exposure portion of the conduction portion is exposed on a bottom surface of the housing recess portion on the front surface.

7. The wearable device according to claim 5, wherein
a housing recess portion configured to house the sensor main body is formed on the front surface of the attachment portion, and
the front surface exposure portion of the conduction portion is exposed on a side surface of the housing recess portion on the front surface.

8. The wearable device according to claim 5, wherein
the attachment portion is a band body configured to extend in circumferential directions of an arm and configured to be attached to the arm, and
the back surface exposure portion and the front surface exposure portion of the conduction portion are disposed at positions overlapping each other in extending directions of the band body.

9. The wearable device according to claim 5, wherein
the attachment portion is a band body configured to extend in circumferential directions of an arm and configured to be attached to the arm, and
the back surface exposure portion and the front surface exposure portion of the conduction portion are disposed at positions not overlapping each other in extending directions of the band body.

10. The wearable device according to claim 9, wherein the conduction portion includes a wiring portion that electrically couples the back surface exposure portion and the front surface exposure portion to each other and is embedded in the band body without being exposed to outside.

11. The wearable device according to claim 1, wherein
the sensor main body includes a plurality of the electrodes, and
the attachment portion includes a plurality of the conduction portions respectively corresponding to the plurality of electrodes.

12. The wearable device according to any claim 1, wherein the detection portion of the sensor main body is a sensor configured to detect an electromagnetic wave or a sound wave from the living body.

13. The wearable device according to claim 12, wherein the detection portion of the sensor main body is an optical pulse wave sensor.

14. A wearable device comprising:
a base body that includes a housing fitting portion and an elongated band configured to encircle a portion of a living body so that a surface of the base body faces towards and is in contact with a surface of the living body;
the housing fitting portion including a recess surrounded by a bottom wall portion of the housing fitting portion and a side wall portion of the housing fitting portion, the side wall portion of the housing fitting portion projecting away from the bottom wall portion of the housing fitting portion towards an open upper end of the recess;
a sensor main body configured to be positioned in the recess of the housing fitting portion, the sensor main body including a top wall, a bottom wall and a side wall extending between the top wall and the bottom wall of the sensor main body so that when the sensor main body is positioned in the recess of the housing fitting portion, the bottom wall of the sensor main body faces the bottom wall portion of the housing fitting portion and the side wall of the sensor main body faces the side wall portion of the housing fitting portion;
the sensor main body including an electrode configured to detect an electrical characteristic of a living body and a detection portion different from the electrode and configured to detect living body information other than the electrical characteristic of the living body, the electrode having an exposed portion that is exposed on the bottom wall of the sensor main body or on the side wall of the sensor main body;
the side wall portion of the housing fitting portion or the bottom wall portion of the housing fitting portion including a conduction portion configured to contact the exposed portion of the electrode of the sensor main body when the sensor main body is positioned in the recess to provide conductivity between the electrode of the sensor main body and the surface of the living body when the wearable device is worn on the living body; and
the base body being configured to not act as a blockage between the detection portion of the sensor main body and the surface of the living body in a conduction state in which the sensor main body is positioned in the recess and the surface of the base body is in contact with the surface of the living body.

15. The wearable device according to any claim 14, wherein the detection portion of the sensor main body is a sensor configured to detect an electromagnetic wave or a sound wave from the living body.

16. The wearable device according to claim 14, wherein the bottom wall portion of the housing fitting portion includes a through opening aligned with the detection portion.

17. The wearable device according to claim 14, wherein the electrode is a first electrode, and further comprising a second electrode and a third electrode each having an exposed portion that is exposed on the bottom wall of the sensor main body or on the side wall of the sensor main body, the conduction portion of the housing fitting portion being a first conduction portion, the housing fitting portion further comprising a second conduction portion configured to contact the exposed portion of the second electrode of the sensor main body when the sensor main body is positioned in the recess and a third conduction portion configured to contact the exposed portion of the third electrode of the sensor main body when the sensor main body is positioned in the recess.

18. The wearable device according to claim 14, wherein the electrode is on the side wall of the sensor main body and projects away from the side wall of the sensor main body, and the conduction portion is an upstanding conduction portion that extends away from the bottom wall portion of the housing fitting portion, the upstanding conduction portion including a recess in which the electrode is positioned.

19. The wearable device according to claim 14, wherein the base body includes a base main body and a base cover, the base cover including the housing fitting portion and the elongated band, the base cover being positioned in overlying relation to the base main body, the conduction portion being formed on the base main body.

20. The wearable device according to claim 14, wherein the conduction portion includes an exposed surface configured to contact the surface of the living body when the wearable device is worn on the living body.
